(19) United States
(12) Patent Application Publication
(10) Pub. No.: US 2026/0296766 A1
(43) Pub. Date: Oct. 1, 2026

(54) AEROSOL PRODUCT WITH COMPRESSED GAS PROPELLANT

(71) Applicant: The Procter & Gamble Company, Cincinnati, OH (US)

(72) Inventor: Shawn David McConaughy, Cincinnati, OH (US)

(21) Appl. No.: 19/630,573

(22) Filed: Mar. 27, 2026

Related U.S. Application Data
(60) Provisional application No. 63/779,952, filed on Mar. 28, 2025.

(51) Int. Cl. B65D 83/48; B65D 83/141

(52) U.S. Cl. CPC B65D 83/48; B65D 83/141

(57) ABSTRACT

An aerosol product with an aerosol dispenser that includes a dispenser container defining a cavity. An adsorbent, a transfer agent, and a compressed gas propellant are within the cavity. A product reservoir containing a product composition is disposed within the cavity. A valve assembly provides fluid communication between the reservoir, the cavity, and an actuator to dispense the product.

### FIELD OF THE INVENTION

The present disclosure generally relates to aerosol products that utilize a non-hydrocarbon propellant, such as compressed gas. The aerosol products can be useful for dispensing product compositions including deodorant and/or antiperspirant, hairspray, dry shampoo, sunscreen, fabric care, air care including air fresheners, hard and soft surface care including oven cleaner, dusting spray, and furniture polish, pesticides such as bug repellant and insecticides, herbicides, cooking spray, and the like.

### BACKGROUND OF THE INVENTION

Aerosol products, including personal care products such as hairsprays, dry shampoos, deodorants, and antiperspirants, are preferred by some consumers because they offer a convenient and effective way to apply these products. Aerosol products release a fine mist that facilitates broad and even coverage. Additionally, the rapid drying time upon application generally results in a non-greasy and non-sticky feel.

Traditionally, many aerosol formulations use hydrocarbon propellants. Hydrocarbon propellants provide a relatively constant pressure inside the canister, which provides a relatively consistent spray rate over the lifetime of the can. However, some consumers would prefer aerosol products that employ non-hydrocarbon propellants, including compressed gases, which are in the gas state at 25° C. and at a pressure of at least 50 psi, such as compressed air, oxygen, nitrogen, carbon dioxide, or mixtures thereof. Developing an aerosol product that is both effective and acceptable to consumers while utilizing a compressed gas propellant presents challenges.

Unlike hydrocarbon propellants, compressed gases remain in a vapor state. Consequently, as the product is dispensed, the pressure within the container decreases, which can make it difficult to maintain the spray rate throughout the lifetime of the can. This aspect is particularly important for personal care compositions, as the efficacy of the product may be adversely affected if the spray rate deviates significantly from the range preferred by consumers.

Therefore, there is a continued need for an improved aerosol product that uses a compressed gas propellant and delivers a consumer acceptable spray rate from the initial use until the product is depleted.

### SUMMARY OF THE INVENTION

An aerosol product comprising an aerosol dispenser comprising: (a) a dispenser container comprising a cavity; wherein an adsorbent, a transfer agent, and a compressed gas propellant are disposed within the cavity; (b) a product reservoir disposed within the cavity; (c) a product composition disposed within the product reservoir; (d) a valve assembly; wherein the valve assembly comprises: (i) a connector disposed in a lower end of the valve assembly; wherein the actuator is in fluid communication with the product reservoir and the cavity; (ii) an actuator disposed in an upper end of the valve assembly; wherein the actuator is in operative communication with the connector; (e) a filter for mitigating escape of the adsorbent from the container.

### BRIEF DESCRIPTION OF THE DRAWINGS

While the specification concludes with claims particularly pointing out and distinctly claiming the subject matter of the present invention, it is believed that the invention can be more readily understood from the following description taken in connection with the accompanying drawings, in which:

FIG. 1 is a side cross-sectional view of a bag-on-valve aerosol dispenser;

FIG. 2 shows the spray rate (g/sec) over time for Aerosol Product Examples A-D;

FIG. 3 shows the can pressure after 1-20 sprays for Aerosol Product Example E, which included a compressed air propellant and an adsorbent;

FIG. 4 shows the cumulative consumption of compressed gas, in this case carbon dioxide (CO2), from the canister;

FIG. 5 shows the canister pressure after 1-20 sprays for Aerosol Product Example F, which included a compressed air propellant, an adsorbent, and a liquid transfer agent;

FIG. 6 shows a comparison of the percentage of pressure drop recovered for Aerosol Product Examples E and F;

FIGS. 7A-C, shows a comparison of the spray rate for examples that include adsorbent only, transfer agent only, and adsorbent and transfer agent across a range of packaging configurations;

FIG. 7D shows the spray rate over time for two examples on with 40 g of activated carbon and another with 40 g activated carbon and 20 g of water;

FIGS. 8A-B show a comparison of the spray rates for different ratios of liquid transfer agent to adsorbent;

FIGS. 9A-C show the spray rate over time for different ratios of liquid transfer agent to adsorbent at different starting pressures for the compressed air inside the can;

FIG. 10 shows a comparison of the spray rate for an example packaging configuration across a range of starting internal canister pressures, 45 psi (310 kPa) to 125 psi (862 kPa);

FIG. 11 shows the spray rate over time for an aerosol product with two different size bags filled with two different amounts of a deodorant composition; and

FIG. 12 is a side cross-sectional view of an inverted bag-on-valve aerosol dispenser.

### DETAILED DESCRIPTION OF THE INVENTION

Some consumers want aerosol products, in particular aerosol personal care products, that have a compressed gas propellant. However, as the product is dispensed, the pressure within the container decreases, which can make it difficult to maintain a consumer acceptable spray rate from the initial use until the product is depleted or at least 100-150 seconds of spraying.

U.S. Pat. No. 12,023,703 (hereinafter “'703 Patent”) discloses one solution that employs bag-on-valve aerosol technology and the inclusion of an adsorbent, such as activated carbon. This combination is intended to help maintain an acceptable spray rate throughout the product's lifespan by using the activated carbon to replenish carbon dioxide (CO2) and sustain the can's pressure.

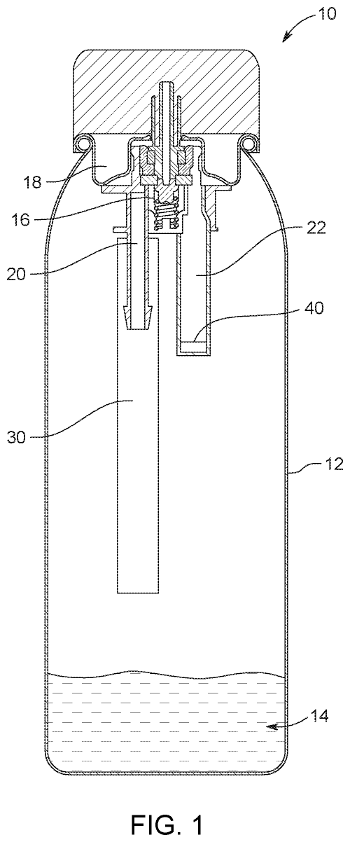

FIG. 1 shows a representation of the teachings of the '703 Patent. FIG. 1 shows an aerosol dispenser 10 that includes a dispenser container 12, such as a can or canister, adsorbent 14, a valve assembly 16, and a mounting cup 18 for both sealing the container and for housing valve assembly 16. A first dip tube 20 and second dip tube 22 extend from valve assembly 16. A distal portion of first dip tube 20 is positioned within a product reservoir 30, such as a bag that can be made from a flexible laminate material. Second dip tube 22 contains a means, such as filter or frit 40, to prevent adsorbent 14 from passing into the valve and being ultimately dispensed along with the consumer product.

The dispenser described the '703 Patent and shown in FIG. 1 was tested with an example deodorant composition, as shown in FIGS. 1-10 and accompanying text. The dispenser container was an aluminum can with a pressure limit of 180 psi (1241.1 kPa) and an internal volume of 230 mL. The can was fitted with a reservoir, specifically a bag-on-valve system. A range of actuators with different exit orifice dimensions were tested (see, described herein), as well as different configurations of gas and liquid channels (see, described herein). During testing, different amounts of compressed CO2 gas were used, varying from 2.5 to 10.5 grams. This produced initial internal pressures in the can that ranged from 45 psi (310.3 kPa) to 125 psi (861.8 kPa). Spray rate tests were conducted according to the Spray Rate Method, described herein, to determine if the aerosol product provided a consumer acceptable spray rate over time. When a liquid transfer agent was present, it was placed inside the can with the adsorbent prior to filling the reservoir and pressurizing the can with compressed gas. The results of these tests are shown in FIGS. 1-10 and accompanying text.

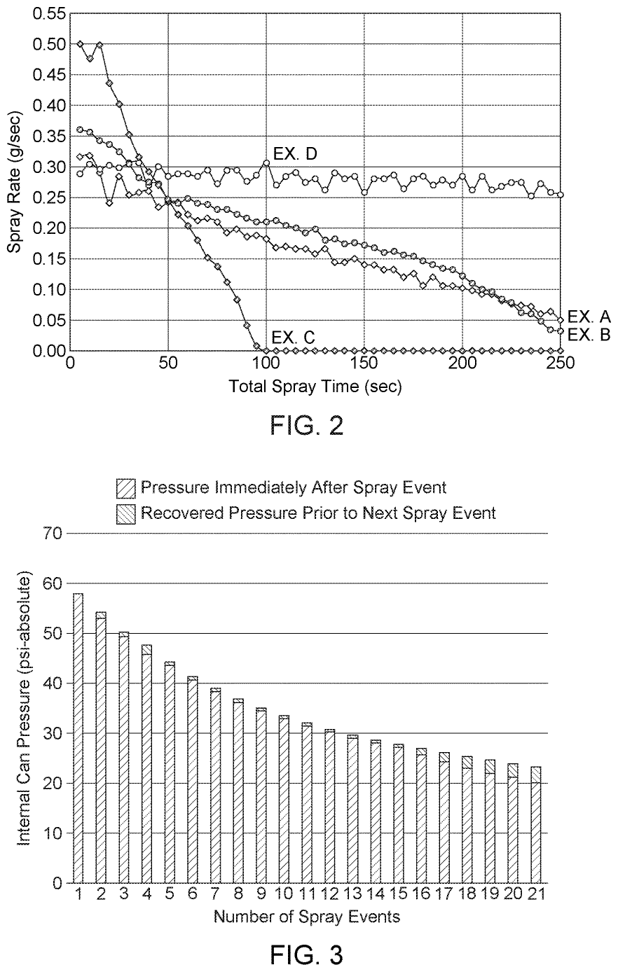

FIG. 2 shows the spray rates for Aerosol Product Examples A, B, C, and D, which are detailed in Table in, below.

TABLE 1
Aerosol Product Examples A-D

| | Ex. A | Ex. B | Ex. C | Ex. D |
|---|---|---|---|---|
| Deodorant Composition | Composition Example 1 | | | Secret ® |
| | (see) | | | Aluminum Free |
| Reservoir | 100 mL Bag-on-valve | | | Dry Spray (made |
| Mass of Deodorant | 60 | | | in January of |
| Composition (g) | | | | 2025. Lot code: |
| Mass of Adsorbent | 40 | 40 | 0 | 5006144501) |
| (Activated Carbon) (g) | | | | with a propellant |
| Mass of Compressed | 7.5 | | | that includes |
| Gas (CO2) (g) | | | | hydrofluoro- |

| | | |
|---|---|---|
| Connector | Connector A (see) | carbon 152a |
| Actuator Example | Actuator A (see) | |

FIG. 2 shows the spray rate (g/sec) over time for Ex. A-D. Ex. D uses a hydrocarbon propellant and had a relatively constant spray rate over 250 seconds of total spray time. Example C, which had a compressed gas propellant and did not contain activated carbon, initially produced the highest spray rate. However, within 100 seconds of use, the spray rate quickly dropped to less than 0.1 g/s. This decline was due to the loss of pressure in the container with each spray, which hindered its ability to expel the remaining product effectively.

Examples A and B, both contain 40 g of activated carbon, performed better than Example C. However, both A and B saw a decrease in their spray rate. Specifically, the spray rate dropped below 0.2 g/s before reaching 150 seconds, fell below 0.1 g/s well before 250 seconds, and ultimately decreased to 0.05 g/s before the 250-second mark. This decline may indicate how quickly the can loses pressure and, consequently, its ability to dispense product effectively.

Ex. A was sprayed approximately every five minutes and Ex. B was sprayed approximately every 24 hours. As shown in FIG. 2, the spray rates for both Ex. A and B are very similar, which indicates that giving the product more time between sprays does not noticeably increase the amount of CO2 that is released from the activated carbon. Ex. A and B offer an improvement over the system without carbon (Ex. C) but does not come close to achieving the spray rate of the liquid propellant system (Ex. D) and to achieve a consumer acceptable product the spray rate needs to be maintained over a longer period. While Examples A and B may offer a sufficient spray rate over for a desirable total spray time, Composition Example 1 has a relatively low viscosity due to its formula containing over 60% ethanol. This data suggests that compositions with higher viscosity—such as antiperspirant or deodorant compositions that contain dipropylene glycol, propylene glycol, and/or antiperspirant actives—are more challenging to dispense. As a result, these thicker compositions are unlikely to achieve a consumer-acceptable spray rate for a desirable total spray time.

FIG. 3 shows the spray rates for Aerosol Product Example E, which are detailed in, below.

TABLE 2
Aerosol Product Example E

| | Ex. E |
|---|---|
| Composition | Composition Example 2 (see) |
| Reservoir | 100 mL Bag-on-valve |
| Mass of Composition (g) | 60 |
| Mass of Adsorbent (Activated Carbon) (g) | 40 |
| Mass of Compressed Gas (CO2) (g) | 7.1 |
| Connector Example | Connector A (see) |
| Actuator Example | Actuator A (see) |

Aerosol Product Example E was tested according to the Spray Rate Method, described herein, but with addition of a data logger that was inserted into the can before sealing and adding compressed gas. The data logger recorded pressure, temperature, and time every 3 seconds over a 4-hour period. During this time, the canister underwent multiple spray rate tests until it was empty. The results indicated that the pressure drop within the canister was significantly greater than the pressure recovery from the CO2 released by the activated carbon. This discrepancy contributed to a significant decline in the spray rate over time.

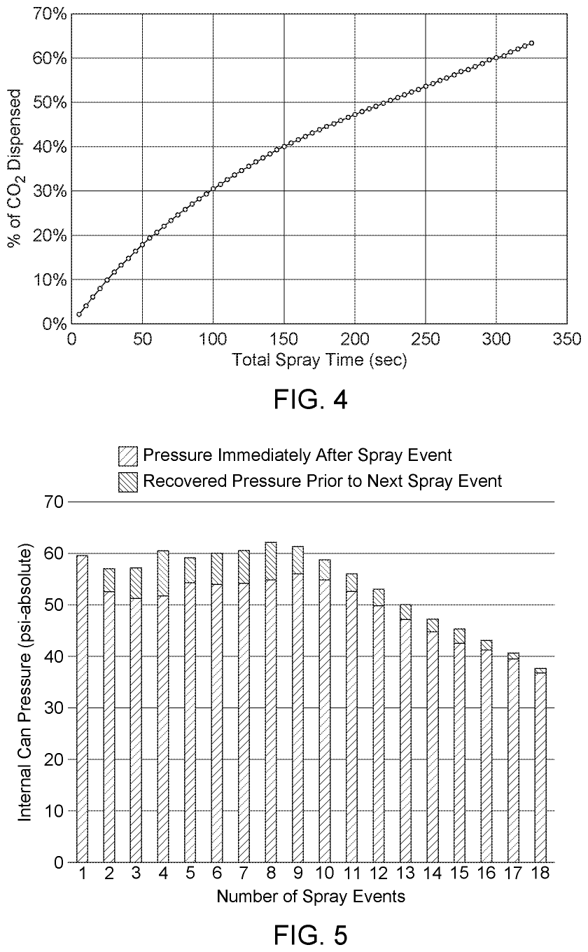

One possible explanation for the pressure drop is that the aerosol product is running out of CO2. However, FIG. 4 shows the cumulative consumption of compressed gas, in this case CO2, from the can over time. This was collected by performing a test that follows the Spray Rate Method, described herein, with the addition step of spraying the aerosol into a bag on every 5^{th} spray to determine the mass of liquid dispensed. The total mass loss, after subtracting the liquid mass remaining in the bag, represents the mass of gas lost from the canister. This data for the gas loss is then analyzed using curve fitting to create a mathematical model. This model is subsequently used to calculate the mass of gas lost with each spray. As shown in FIG. 4, only 60% of the CO2 initially added to the canister is released during the 300 seconds of spray life. The remaining CO2 stayed in the can, with most of it being absorbed by the activated carbon inside. This demonstrated that there is additional CO2 in the can that could be utilized to maintain the internal can pressure if it could be effectively released from the adsorbent, in this case activated carbon.

It was found that by introducing a liquid transfer agent, the compressed gas could be more effectively removed from the adsorbent, converting it into gas. This process helped to repressurize the can thereby improving the spray rate.

FIG. 5 shows the spray rates for Aerosol Product Example F, which are detailed in, below.

TABLE 3
Aerosol Product Example F

| | Ex. F |
|---|---|
| Composition | Composition Example 2 (see) |
| Reservoir | 100 mL Bag-on-valve |
| Mass of Composition (g) | 60 |
| Mass of Adsorbent (Activated Carbon) (g) | 40 |
| Mass of Liquid Transfer Agent (Water) (g) | 20 |
| Mass of Compressed Gas (CO2) (g) | 7.1 |
| Connector Example | Connector A (see) |
| Actuator Example | Actuator A (see) |

FIG. 5 shows the can pressure after 1-20 sprays for an aerosol dispenser, where a liquid transfer agent, in this instance water, is added to the canister with the activated carbon. It was found that the transfer agent enhanced the transfer of CO2 from the activated carbon into the gas phase of the canister. FIG. 5 shows that adding the transfer agent helped maintain a higher canister pressure for a greater number of sprays, as compared to the Aerosol Product Example E in FIG. 3. This can lead to a more consistent spray rate from the beginning to the last use.

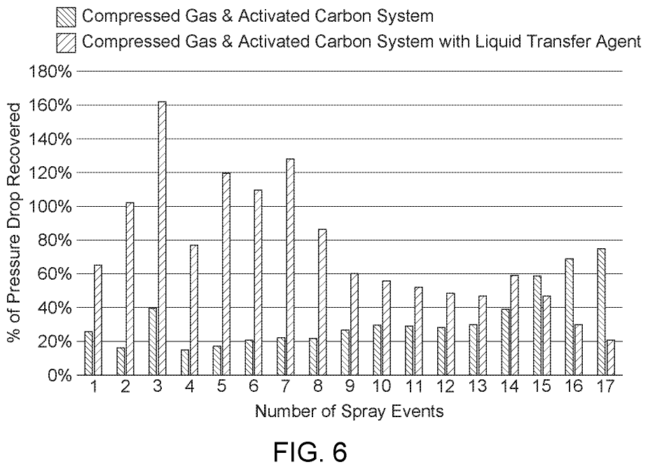

FIG. 6 shows a comparison of the percentage of pressure drop recovered for a compressed gas system with activated carbon (Aerosol Product Example E), as shown in FIG. 3, and a compressed gas system with activated carbon and a liquid transfer agent (Aerosol Product Example F), as shown in FIG. 5. The data in FIG. 6 was determined by taking the pressure increase after a spray event and dividing that by the decrease in pressure that occurred during spraying. As shown in FIG. 6, the system with the addition of a liquid transfer agent typically recovers greater than 50% of the pressure lost between spray events, and across the first 20 sprays averages a recovery of 60%, while the system without a liquid transfer agent more typically recovers only 30% of the lost pressure, over the first 20 sprays averaging 33% recovery.

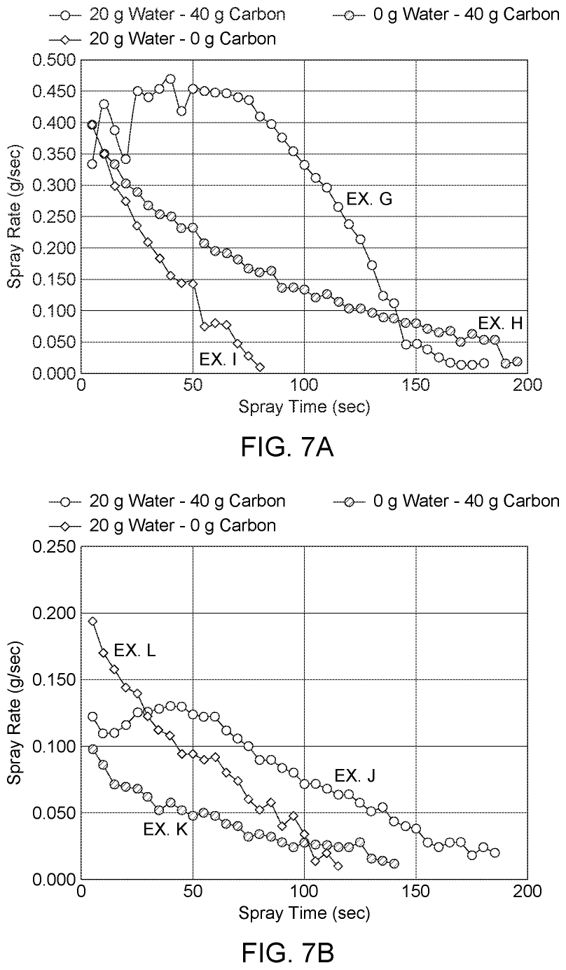

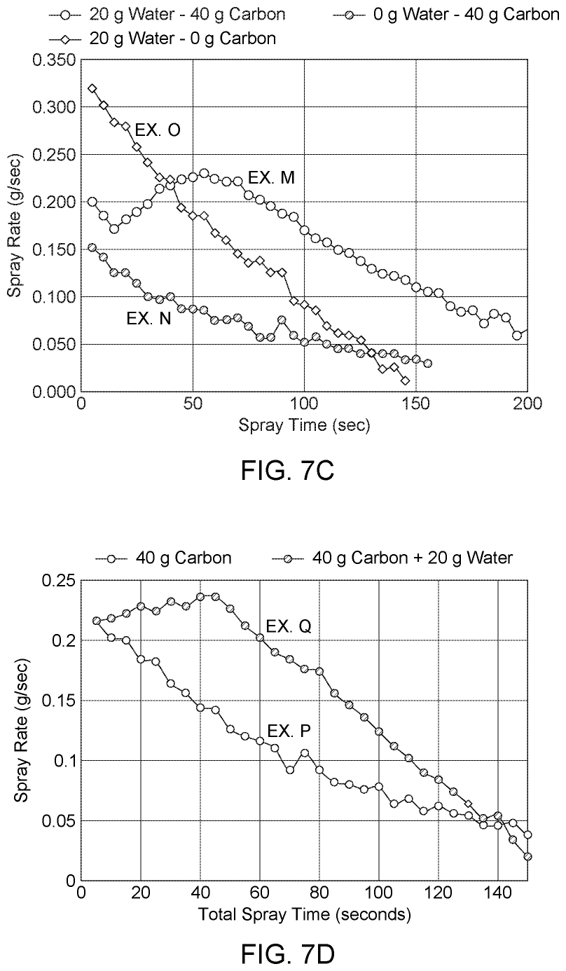

FIG. 7A shows the spray rates for Aerosol Products G, H, and I, which are detailed in, below.

TABLE 4
Aerosol Product Examples G-I

| | Ex. G | Ex. H | Ex. I |
|---|---|---|---|
| Composition | Composition Example 1 (see) | | |
| Reservoir | 100 mL Bag-on-Valve | | |
| Mass of Composition | 60 | | |
| (g) | | | |
| Mass of Adsorbent | 40 | 40 | 0 |
| (Activated Carbon) | | | |
| (g) | | | |
| Mass of Liquid | 20 | 0 | 20 |
| Transfer Agent | | | |
| (Water) (g) | | | |
| Mass of Compressed | 7.25 | 4.89 | 2.64 |
| Gas (CO2) (g) | | | |

| | |
|---|---|
| Connector Example | Connector A (see) |
| Actuator Example | Actuator A (see) |

FIG. 7B shows the spray rates for Aerosol Products J, K, and L, which are detailed in, below.

TABLE 5
Aerosol Product Examples J-L

| | Ex. J | Ex. K | Ex. L |
|---|---|---|---|
| Composition | Composition Example 1 (see) | | |
| Reservoir | 100 mL Bag-on-Valve | | |
| Mass of Composition | 60 | | |
| (g) | | | |
| Mass of Adsorbent | 40 | 40 | 0 |
| (Activated Carbon) | | | |
| (g) | | | |
| Mass of Liquid | 20 | | |
| Transfer Agent | | | |
| (Water) (g) | | | |
| Mass of Compressed | 4.8 | 7.26 | 2.58 |
| Gas (CO2) (g) | | | |

| | |
|---|---|
| Connector Example | Connector A (see) |
| Actuator Example | Actuator C (see) |

FIG. 7C shows the spray rates for Aerosol Products M, N, and O, which are detailed in, below.

TABLE 6
Aerosol Product Examples M-O

| | Ex. M | Ex. N | Ex. O |
|---|---|---|---|
| Composition | Composition Example 1 (see) | | |
| Reservoir | 100 mL Bag-on-Valve | | |
| Mass of Composition | 50 | | |
| (g) | | | |
| Mass of Adsorbent | 40 | 40 | 0 |
| (Activated Carbon) | | | |
| (g) | | | |
| Mass of Liquid | 20 | | |
| Transfer Agent | | | |
| (Water) (g) | | | |
| Mass of Compressed | 4.83 | 7.23 | 2.61 |
| Gas (CO2) (g) | | | |

| | |
|---|---|
| Connector Example | Connector B (see) |
| Actuator Example | Actuator C (see) |

FIGS. 7A-C compared the spray rates of different aerosol products with compressed gas propellant across various packaging configurations. In FIGS. 7A-C, the aerosol product that contained an adsorbent (40 g activated carbon) and a liquid transfer agent (20 g water) exhibited an increased spray rate versus the aerosol product without liquid transfer agent (only 40 g activated carbon). As shown in FIGS. 7A-C, the spray rate of the aerosol product with the liquid transfer agent in combination with the adsorbent is typically 2-3 times higher than the example without the liquid transfer agent over the majority of the product's lifespan. Additionally, the transfer agent (20 g water) in combination with the adsorbent (40 g activated carbon), maintained an elevated spray rate over time, as compared to the aerosol product with only the liquid transfer agent (20 g water) which exhibited a rapid decline in spray rate over time.

FIG. 7D shows the spray rates for Aerosol Products P and Q which are detailed in, below.

TABLE 7
Aerosol Product Examples P and Q

| | |
|---|---|
| Ex. P | Ex. Q |

| | |
|---|---|
| Composition | Composition Example 2 (see) |
| Reservoir | 100 mL Bag-on-valve |
| Mass of Composition (g) | 50 |
| Mass of Adsorbent (Activated | 40 |

| | | |
|---|---|---|
| Carbon) (g) | | |
| Mass of Liquid Transfer | 0 | 20 |
| Agent (Water) (g) | | |
| Mass of Compressed Gas | 7.12 | 5.1 |
| (CO2) (g) | | |

| | |
|---|---|
| Connector Example | Connector A (see) |
| Actuator Example | Actuator A (see) |

FIG. 7D compared the spray rate of an aerosol products with compressed gas propellants and a higher viscosity deodorant composition, as compared to Aerosol Product Examples M-O. Example Q, which included 40 g adsorbent (activated carbon) and 20 g liquid transfer agent (water), maintained a higher spray rate for longer, as compared to Example P, which did not include a liquid transfer agent.

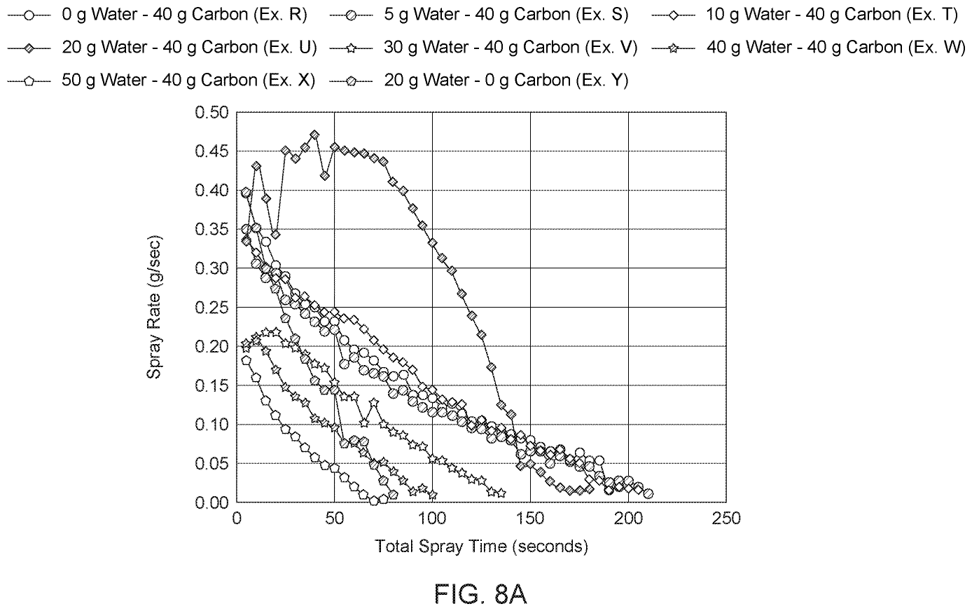

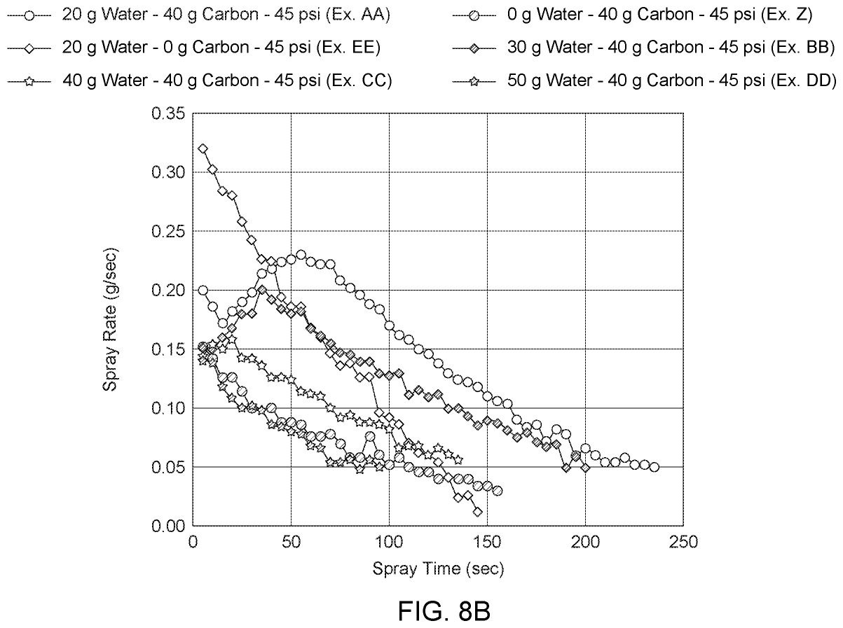

FIG. 8A shows the spray rates for Aerosol Products R-Y, which are detailed in and, below.

TABLE 8
Aerosol Product Examples R-U

| | Ex. R | Ex. S | Ex. T | Ex. U |
|---|---|---|---|---|
| Composition | Composition Example 1 (see) | | | |
| Reservoir | 100 mL Bag-on-valve | | | |
| Mass of Composition (g) | 50 | | | |
| Mass of Adsorbent (Activated | 40 | | | |
| Carbon) (g) | | | | |
| Mass of Liquid Transfer | 0 | 5 | 10 | 20 |
| Agent (Water) (g) | | | | |
| Ratio of liquid transfer agent | 0:1 | 5:4 | 1:4 | 1:2 |
| to adsorbent | | | | |
| Mass of Compressed Gas | 7.25 | 6.6 | 5.88 | 4.89 |
| (CO2) (g) | | | | |

| | |
|---|---|
| Connector Example | Connector A (see) |
| Actuator Example | Actuator A (see) |

TABLE 9
Aerosol Product Examples V-Y

| | Ex. V | Ex. W | Ex. X | Ex. Y |
|---|---|---|---|---|
| Composition | Composition Example 1 (see) | | | |
| Reservoir | 100 mL Bag-on-valve | | | |
| Mass of Composition (g) | 50 | | | |
| Mass of Adsorbent (Activated | 40 | | | 0 |
| Carbon) (g) | | | | |
| Mass of Liquid Transfer | 30 | 40 | 50 | 20 |
| Agent (Water) (g) | | | | |
| Ratio of liquid transfer agent | 3:4 | 1:1 | 5:4 | |
| to adsorbent | | | | |
| Mass of Compressed Gas | 4.02 | 2.99 | 2.12 | 2.64 |
| (CO2) (g) | | | | |

| | |
|---|---|
| Connector Example | Connector A (see) |
| Actuator Example | Actuator A (see) |

FIG. 8B shows the spray rates for Aerosol Products Z-EE, which are detailed in, below.

TABLE 10
Aerosol Product Examples Z-EE

| | Ex. Z | Ex. AA | Ex. BB | Ex. CC | Ex. DD | Ex. EE |
|---|---|---|---|---|---|---|
| Composition | Composition Example 1 (see) | | | | | |
| Reservoir | 100 mL Bag-on-valve | | | | | |
| Mass of Composition (g) | 50 | | | | | |
| Mass of Adsorbent | 40 | | | | | 0 |
| (Activated Carbon) (g) | | | | | | |
| Mass of Liquid Transfer Agent | 0 | 20 | 30 | 40 | 50 | 20 |
| (Water) (g) | | | | | | |
| Ratio of liquid transfer agent | 0:1 | 1:2 | 3:4 | 1:1 | 5:4 | |
| to adsorbent | | | | | | |
| Mass of Compressed Gas (CO2) (g) | 7.26 | 4.8 | 4.04 | 3.07 | 2.1 | 2.58 |

| | |
|---|---|
| Connector Example | Connector A (see) |
| Actuator Example | Actuator C (see) |

FIGS. 8A-B shows the spray rate over time for examples with ratios of liquid transfer agent to adsorbent that range from 1:4 to 5:4 with a first control that includes 0 g water and 40 g carbon and a second control that includes 20 g water and 0 g carbon. Both FIGS. 8A and 8B show that the compositions that maintained a spray rate above 0.1 g/s the longest had a ratio of liquid transfer agent to adsorbent of about 1:2 or 1:4 and the examples that included both liquid transfer agent and adsorbent that performed the worst had a ratio of liquid transfer agent to adsorbent of 1:1 or 5:4.

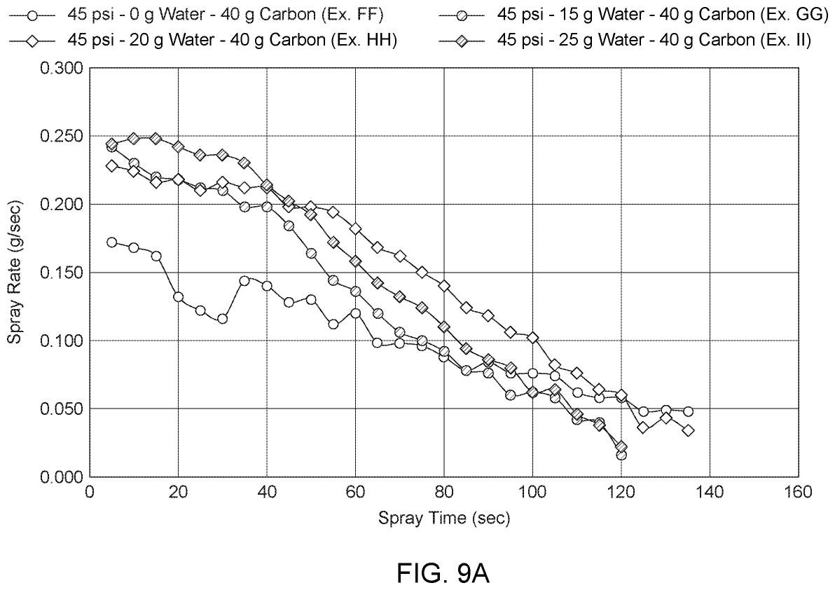

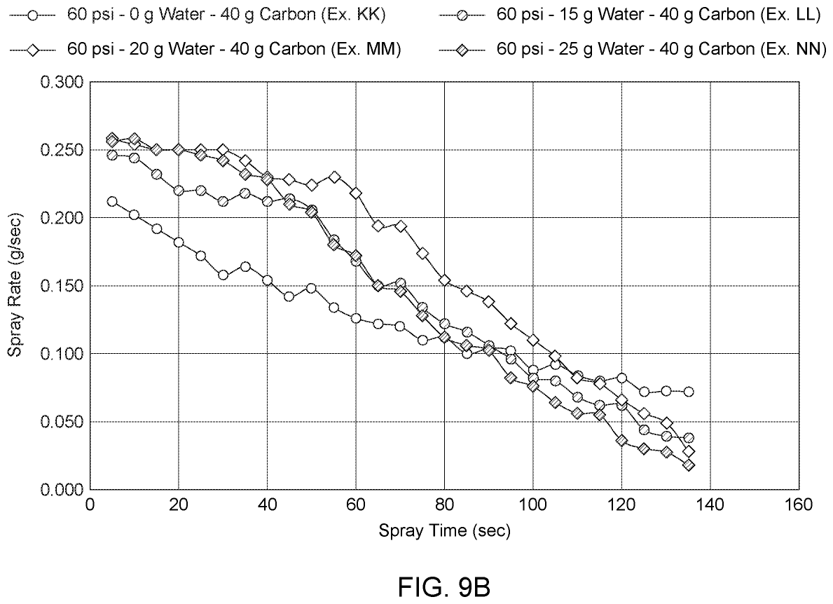

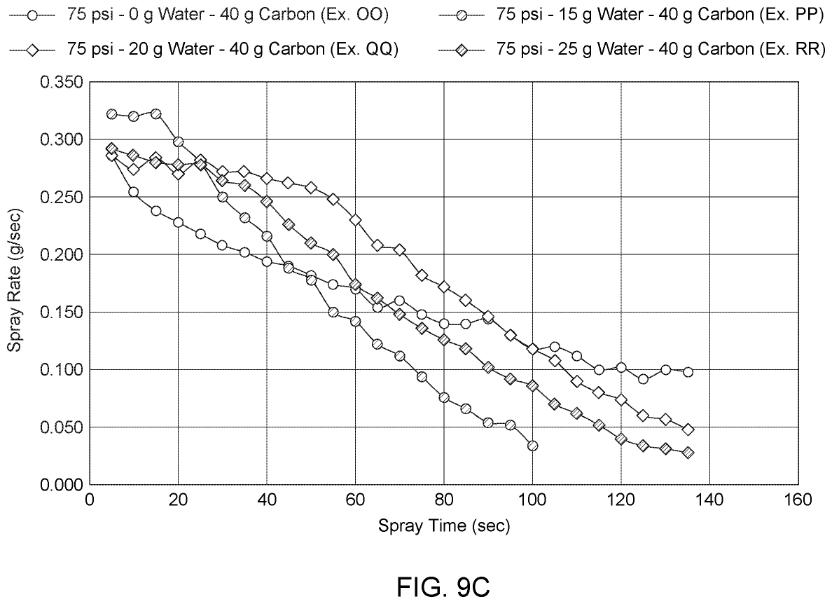

FIG. 9A shows the spray rates for Aerosol Products FF-II, which are detailed in, below.

TABLE 11
Aerosol Products FF-II

| | Ex. FF | Ex. GG | Ex. HH | Ex. II |
|---|---|---|---|---|
| Composition | Composition Example 1 (see) | | | |
| Fill Pressure | 45 psi (310 kPa) | | | |
| Reservoir | 100 mL Bag-on-valve | | | |
| Mass of Composition | 50 | | | |
| (g) | | | | |
| Mass of Adsorbent | 40 | | | |
| (Activated Carbon) (g) | | | | |
| Mass of Liquid | 0 | 15 | 20 | 25 |
| Transfer Agent (Water) | | | | |
| (g) | | | | |
| Ratio of liquid transfer | 0:1 | 3:8 | 1:2 | 5:8 |
| agent to adsorbent | | | | |
| Mass of Compressed | 7.22 | 5.23 | 4.9 | 4.32 |
| Gas (CO2) (g) | | | | |

| | |
|---|---|
| Connector Example | Connector A (see) |
| Actuator Example | Actuator C (see) |

FIG. 9B shows the spray rates for Aerosol Products KK-NN, which are detailed in, below.

TABLE 12
Aerosol Products KK-NN

| | | | |
|---|---|---|---|
| Ex. KK | Ex. LL | Ex. MM | Ex. NN |

| | |
|---|---|
| Composition | Composition Example 1 (see) |
| Fill Pressure | 60 psi (414 kPa) |
| Reservoir | 100 mL Bag-on-valve |
| Mass of | 50 |

Composition (g)

| | |
|---|---|
| Mass of | 40 |

| | | | | |
|---|---|---|---|---|
| Adsorbent | | | | |
| (Activated | | | | |
| Carbon) (g) | | | | |
| Mass of Liquid | 0 | 15 | 20 | 25 |
| Transfer Agent | | | | |
| (Water) (g) | | | | |
| Ratio of liquid | 0:1 | 3:8 | 1:2 | 5:8 |
| transfer agent to | | | | |
| adsorbent | | | | |
| Mass of | 7.82 | 5.78 | 5.3 | 4.76 |
| Compressed Gas | | | | |
| (CO2) (g) | | | | |

| | |
|---|---|
| Connector | Connector B (see) |

Example

| | |
|---|---|
| Actuator | Actuator A (see) |

Example

FIG. 9C shows the spray rates for Aerosol Products OO-RR, which are detailed in, below.

TABLE 13
Aerosol Products OO-RR

| | | | |
|---|---|---|---|
| Ex. OO | Ex. PP | Ex. QQ | Ex. RR |

| | |
|---|---|
| Composition | Composition Example 1 (see) |
| Fill Pressure | 75 psi (517 kPa) |
| Reservoir | 100 mL Bag-on-valve |
| Mass of | 50 |

Composition (g)

| | |
|---|---|
| Mass of | 40 |

| | | | | |
|---|---|---|---|---|
| Adsorbent | | | | |
| (Activated | | | | |
| Carbon) (g) | | | | |
| Mass of Liquid | 0 | 15 | 20 | 25 |
| Transfer Agent | | | | |
| (Water) (g) | | | | |
| Ratio of liquid | 0:1 | 3:8 | 1:2 | 5:8 |
| transfer agent to | | | | |
| adsorbent | | | | |
| Mass of | 8.57 | 6.55 | 5.91 | 5.17 |
| Compressed Gas | | | | |
| (CO2) (g) | | | | |

| | |
|---|---|
| Connector | Connector B (see) |

Example

| | |
|---|---|
| Actuator | Actuator A (see) |

Example

FIGS. 9A-C show the spray rate over time for examples where the can is filled to a pressure of 45 psi (310 kPa), 60 psi (414 kPa), and 75 psi (517 kPa), respectively and with ratios of liquid transfer agent to adsorbent that range from 1:2 to 3:8 with a control that includes 0 g water and 40 g carbon. In FIGS. 9A and 9B, all three examples that included a liquid transfer agent maintained a spray rate above 0.2 g/s and 0.1 g/s longer than the control. In FIG. 9C, all three examples that included a liquid transfer agent maintained a spray rate above 0.2 g/s for longer than the control and Examples OO, QQ, and RR maintained a spray rate of over 0.1 g/s for a longer duration than similar samples that started with a lower initial pressure. Example PP was identified as a potential outlier in FIG. 9C. It exhibited a higher initial starting pressure and spray rate compared to the other examples and it experienced a more rapid decline in performance. This suggests that the actuator or another piece may have been too large, leading to a quicker loss of pressure. It is anticipated that if Example PP were to be rerun, its performance would align more closely with those examples.

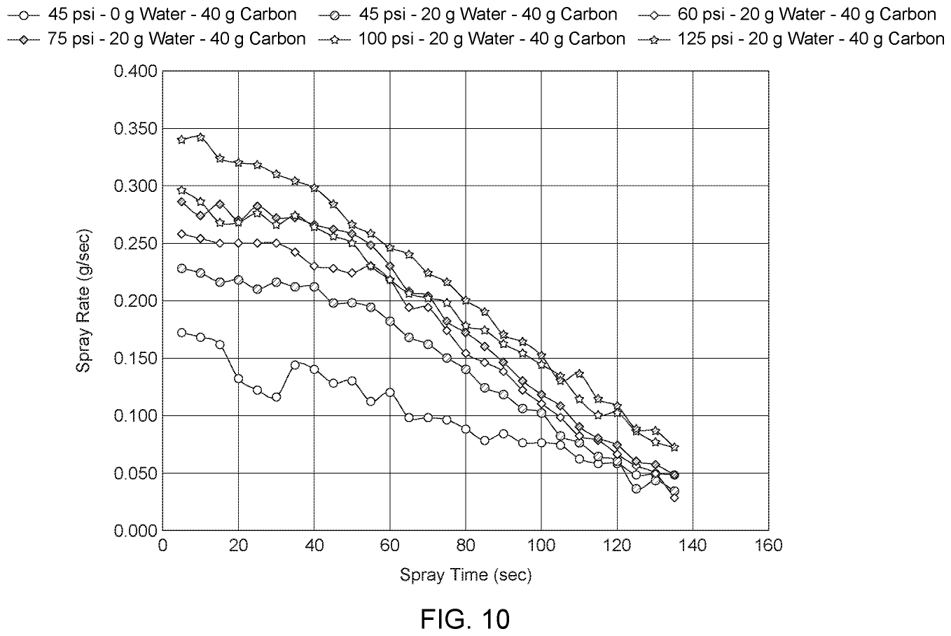

FIG. 10 shows the spray rates for the following products:
Ex. FF: Pressurized to 45 psi, with a reservoir containing 0 grams of liquid transfer agent (water) and 40 grams of activated carbon adsorbent. (See)
Ex. HH: Pressurized to 45 psi, with a reservoir containing 20 grams of liquid transfer agent (water) and 40 grams of activated carbon adsorbent. (See)
Ex. MM: Pressurized to 60 psi, with a reservoir containing 20 grams of liquid transfer agent (water) and 40 grams of activated carbon adsorbent. (See)
Ex. QQ: Pressurized to 75 psi, with a reservoir containing 20 grams of liquid transfer agent (water) and 40 grams of activated carbon adsorbent. (See)

FIG. 10 also includes the spray rates for Aerosol Products SS-TT, which are detailed in, below.

TABLE 14
Aerosol Products SS-TT

| | |
|---|---|
| Ex. SS | Ex. TT |

| | |
|---|---|
| Composition | Composition Example 1 (see) |

| | | |
|---|---|---|
| Fill Pressure | 100 psi (689 kPa) | 125 psi (862 kPa) |

| | |
|---|---|
| Reservoir | 100 mL Bag-on-Valve |
| Mass of Composition (g) | 50 |
| Mass of Adsorbent | 40 |

(Activated Carbon) (g)

| | |
|---|---|
| Mass of Liquid Transfer | 20 |

Agent (Water) (g)

| | |
|---|---|
| Ratio of liquid transfer | 1:2 |

| | | |
|---|---|---|
| agent to adsorbent | | |
| Mass of Compressed | 7.2 | 9.07 |
| Gas (CO2) (g) | | |

| | |
|---|---|
| Connector Example | Connector B (see) |
| Actuator Example | Actuator A (see) |

FIG. 10 shows the spray rate over time for examples using 20 grams of liquid transfer agent and 40 grams of adsorbent, resulting in a 1:2 ratio of transfer agent to adsorbent. The tests were conducted at various fill pressures, ranging from 45 psi (310 kPa) to 125 psi (862 kPa). The data shows that a higher fill pressure leads to a higher spray rate, and examples with greater fill pressures maintain spray rates above 0.2 g/s and above 0.1 g/s for a longer duration. However, filling the can beyond 125 psi (862 kPa) can exceed its upper safety limit. Cans are typically rated for a maximum pressure of 180 psi (1241 kPa). To ensure safety, the product undergoes a hot water bath test at 55° C. This test can increase the pressure inside the can, simulating conditions that may occur during shipping and storage in non-climate-controlled environments.

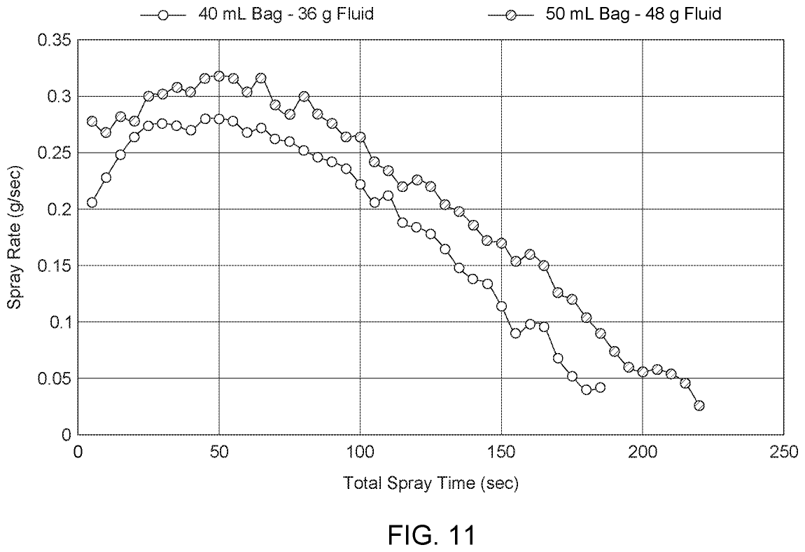

FIG. 11 shows the spray rates for Aerosol Products UU-VV, which are detailed in Table 15, below.

TABLE 15
Aerosol Products UU-VV

| | Ex. UU | Ex. VV |
|---|---|---|
| Composition | Composition Example 3 (see) | |
| Fill Pressure | 75 psi (517 kPa) | |
| Reservoir | 40 mL Bag-on-Valve | 50 mL Bag-on-Valve |
| Mass of Composition (g) | 36 | 45 |
| Mass of Adsorbent | 40 | |
| (Activated Carbon) (g) | | |
| Mass of Liquid | 20 | |
| Transfer Agent | | |
| (Water) (g) | | |
| Mass of Compressed | 6.57 | 6.17 |
| Gas (CO2) (g) | | |

| | |
|---|---|
| Connector Example | Connector A (see) |
| Actuator Example | Actuator B (see) |

FIG. 11 shows the spray rate over time for two aerosol products that contained 20 g water and 40 g activated carbon with different sizes of reservoirs filled with different amounts of the composition. In FIG. 11, both examples had a spray rate greater than 0.2 g/s at 100 s and a spray rate greater than 0.1 g/s at 150 s. It was found that the reservoir size can impact the pressure needed to dispense the liquid and it can be better to have a reservoir size that is appropriate for the amount of liquid being dispensed. The reservoir can be filled at least 70% with the composition, alternatively at least 75%, alternatively at least 80%, alternatively at least 85%, and alternatively at least 90%. It was also found that a reservoir that has flat seals, without gussets, can also aid in dispensing the product.

### Aerosol Dispenser

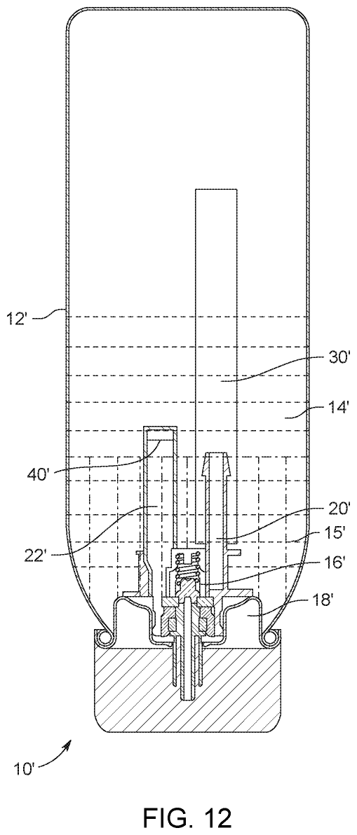

The aerosol dispenser can be any suitable aerosol dispenser, including the dispenser shown in FIG. 1 and described in the accompanying text (which is not inverted) and an inverted dispenser, as shown in FIG. 12. FIG. 12 shows an aerosol dispenser 10′ that includes a dispenser container 12′, adsorbent 14′, a liquid transfer agent 15′, a valve assembly 16′ located at the bottom portion of the aerosol container, and a mounting cup 18′ for both sealing the container and for housing valve assembly 16′. A first dip tube 20′ and second dip tube 22′ extend from valve assembly 16′. A distal portion of first dip tube 20′ is positioned within a product reservoir 30′ and it can extend further into the reservoir than the adsorbent 14′ and/or the liquid transfer agent 15′. Second dip tube 22′ contains a means, such as filter or frit 40′, to prevent adsorbent 14′ from passing into the valve and being ultimately dispensed along with the consumer product.

The aerosol dispenser may include a connector that links the reservoir to the actuator and transfers the product composition from the bag and the compressed gas from the cavity to the actuator when the actuator is pressed. The connector can include a liquid orifices and a gas orifice. The flow rate and the spray pattern can be impacted by the design of the connector and actuator. The ratio of the area of the liquid orifice to the area of the gas orifice can be ≤1:1, alternatively ≤3:4, alternatively ≤2:3, alternatively ≤1:2. The ratio of the area of the liquid orifice diameter(s) to the area of the gas orifice diameter(s) can be ≥1:5, ≥1:4, ≥1:3, and ≥1:2.

The aerosol dispenser may include a pressurized can having a cavity defined by an interior wall of the can. The interior wall may optionally include a lining on all or a portion of an interior surface of the can. The lining may be a thin layer of epoxy resin or BPA-free alternatives like polyethylene-based liners, polyamide-based liners, and/or plant-based coatings, all of which are intended to protect the can from corrosion or from reacting with the adsorbent or transfer agent. A compressed air propellant, an adsorbent, and a liquid transfer agent maybe disposed within the cavity.

The reservoir can be displaced in the cavity. Although not limited to such, the product reservoirs can contain about 20 g to about 250 g of product composition, alternatively from about 30 g to about 200 g, alternatively from about 35 g to about 100 g, alternatively from about 40 g to about 60 g. Other product reservoir capacities are contemplated herein depending on the application of the aerosol product.

The pressurized can may have a volume from about 100 cm³ to about 500 cm³, alternatively from about 150 cm³ to about 300 cm³, and alternatively from about 170 cm³ to about 230 cm³.

The aerosol dispenser can dispense consumer products in a stream, spray, gel, and/or a foam. The aerosol dispenser can dispense the product as a spray that includes a plurality of particles having a distribution of particle sizes, where the distribution can be quantified in terms of the size at which 50% of the distribution is below a particular size, this is commonly referred to as Dv50. The spray can include particles having a Dv50 from about 20 microns to about 140 microns, or from about 30 microns to about 60 microns. Additionally, it can be advantageous to have a small fraction of large particles, to create an even coating on skin. As such the Dv90, or size which 90% of a particular size, may be from about 60 microns to about 140 microns, or from about 60 microns to 90 microns. Additionally, to reduce inhalation risk, it is beneficial for only a small percentage of particles to be smaller than 10 microns, for instance less than 15% of the total volume of particles is under 10 microns in size, alternatively less than 10%, and alternatively less than 5%. A Malvern Spraytec instrument can be used to evaluate aerosol droplet sizes. The Malvern Spraytec instrument uses a technique of laser diffraction for measurement of the size of the spray droplets. The intensity of light scattered as a laser beam passes through a spray is measured. This data is then analyzed to calculate the size of the droplets that created the scattering pattern. A Malvern Spraytec 2000 is used according to the manufacturer's instructions, with the test samples at a temperature between 20° C. to 22° C.

To be consumer acceptable, the spray rate can be greater than 0.10 g/s for more than 100 seconds of total spray time, according to the Spray Rate Method, described herein. This rate can be maintained for the entire lifetime of the aerosol product, which can exceed 150 seconds of total spray time or even exceeds 200 seconds of total spray time. The spray rate can also be greater than 0.2 g/s for ≥50 s according to the Spray Rate Method, described herein, alternatively ≥60 s, alternatively ≥70 s, alternatively ≥80 s, alternatively ≥90 s, alternatively ≥100 s, alternatively ≥110 s, and alternatively ≥120 s. The spray rate can also be greater than 0.1 g/s for ≥100 s according to the Spray Rate Method, described herein, alternatively ≥110 s, alternatively ≥120 s, alternatively ≥130 s, alternatively ≥140 s, alternatively ≥150 s, alternatively ≥160 s, and alternatively ≥170 s. The spray rate can also be greater than 0.05 g/s for ≥125 s according to the Spray Rate Method, described herein, alternatively ≥140 s, alternatively ≥150 s, alternatively ≥160 s, alternatively ≥170 s. It was found that a spray rate of 0.05 g/sec is so low that many consumers will consider this insufficient for a properly working aerosol, as such a lower limit of 0.05 g/sec will be considered the end of the spray life of a product.

The aerosol dispenser can have a fill pressure of from about 45 psi (310 kPa) to about 180 psi (1241 kPa), alternatively from about 45 psi (310 kPa) to about 150 psi (1034 kPa), alternatively from about 60 psi (414 kPa) to about 125 psi (862 kPa), and alternatively from about 75 psi (517 kPa) to about 100 psi (689 kPa).

The aerosol dispenser can dispense ≥60% of the mass of the product composition from the reservoir, alternatively ≥65%, alternatively ≥70%, alternatively ≥75%, alternatively ≥80%, and alternatively ≥85%.

### Adsorbent

The adsorbent can be any material that can effectively adsorb the compressed gas propellant. The adsorbent can be activated carbon, zeolites, metal-organic frameworks, porous silica, amine-functionalized materials, biochar, graphene-based materials, or combinations thereof.

The aerosol product can include from about 2.5 g to about 120 g of the adsorbent, alternatively from about 15 g to about 100 g, alternatively from about 30 g to about 80 g, alternatively from about 30 g to about 60 g, alternatively from about 35 g to about 50 g. The aerosol product can include ≥10 g of the adsorbent, alternatively ≥20 g, alternatively ≥30 g, alternatively ≥60 g, and alternatively ≥90 g. The aerosol product can include from about 5 g to about 150 g of adsorbent, alternatively from about 20 g to about 140 g, alternatively from about 40 g to about 120 g, alternatively from about 50 g to about 110 g, and alternatively from about 60 g to about 100 g.

### Transfer Agent

The transfer agent can be any material that is able to solubilize the gas adsorbed by the adsorbent. Transfer agents can be liquids at room temperature (20 to 25° C.) and pressures from atmospheric pressure up to 180 psi. Transfer agents can have a gas solubility, such as CO2 or nitrogen, of alternatively ≥0.5 g/L at 25° C., alternatively ≥0.75 g/L at 25° C. alternatively ≥1 g/L at 25° C., alternatively ≥1.1 g/L at 25° C., alternatively ≥1.2 g/L at 25° C., alternatively ≥1.3 g/L at 25° C., and alternatively ≥1.4 g/L at 25° C.

Transfer agents can have polar functional groups or both polar and non-polar functional groups. Transfer agents can contain polar liquids like ketones, ethers, alcohols, and water which increase gas solubility. Transfer agents may include glycerin, polyethylene oxide, propanediol, butylene glycol, dipropylene glycol, propylene glycol, water, and mixtures thereof. Transfer agents can include non-volatile hydrocarbons, including alkanes, esters, triglycerides, and mixtures thereof. The transfer agent can be a liquid emollient, as described hereafter.

The transfer agent can be a silicone oil including polysiloxanes, polyalkyl siloxanes, polyaryl siloxanes, polyalkylaryl siloxanes, polyether siloxane copolymers, and mixtures thereof. Examples of silicone oils can also include Cyclopentasiloxane, Dimethicone and Dimethiconol blend (one example is a material blend sold by Dow Corning by the name “XIAMETER PMX—1503 FLUID), Cyclopentasiloxane and Dimethicone Cross (one example is a material blend sold by Dow Corning by the name “Dow Corning 9045 Silicone Elastomer Blend).

A preservative, antioxidant, and/or chelant, as described below, may be added with the transfer agent.

The aerosol product can include about 2 g to about 60 g of the transfer agent, alternatively from about 7 g to about 45 g, alternatively from about 12 g to about 25 g, alternatively from about 15 g to about 25 g, alternatively from about 17 g to about 23 g, and alternatively from about 19 g to about 21 g. The aerosol product can include ≥5 g of the transfer agent, alternatively ≥10 g, and alternatively ≥15 g.

The weight ratio of the liquid transfer agent to adsorbent can be ≥1:5, alternatively ≥1:4, alternatively ≥1:3, alternatively ≥1:2. The weight ratio of the transfer agent to adsorbent can be ≤1:1, alternatively ≤7:8, alternatively ≤5:6, alternatively ≤4:5, and alternatively ≤3:4. The weight ratio of the liquid transfer agent to adsorbent can be from about 1:6 to less than 1:1, alternatively from about 1:5 to about 3:4, alternatively from about 1:4 to about 2:3, alternatively from about 1:4 to about 1:2, and alternatively from about 1:3 to about 1:2.

### Compositions

The aerosol products can be useful for dispensing product compositions including deodorant and/or antiperspirant, hairspray, dry shampoo, sunscreen, fabric care, air care including air fresheners, hard and soft surface care including oven cleaner, dusting spray, and furniture polish, pesticides such as bug repellant and insecticides, herbicides, cooking spray, and the like. The product composition can be disposed within the product reservoir.

The product composition can be a deodorant and/or antiperspirant composition, which will now be described. Exemplary deodorant and/or antiperspirant compositions can be single-phase compositions or have two or more phases such as suspensions or emulsions. The compositions can be Newtonian or shear thinning fluids. Newtonian fluids can range in viscosity from 5.0 PaS to 0.0005 PaS, measured as the average viscosity over a shear rate range of 0.10 to 100 s−1. The viscosity of shear thinning fluids can be between 50-500 PaS at a shear rate of 0.10 s−1 and 0.05 to 5.0 PaS seconds at a shear rate of 10 s−1, as measure over a shear rate range of 0.10 s−1 to 100 s−1.

The aerosol product can have a weight ratio of adsorbent to product composition of at least 5:9, alternatively at least 4:5, alternatively at least 1:1, alternatively at least 6:5, alternatively at least 7:5, alternatively at least 3:2, alternatively at least 7:4, and alternatively at least 2:1. The aerosol product can have a weight ratio of adsorbent to product composition from about 3:5 to about 3:1, alternatively from about 4:5 to about 5:2, alternatively from about 1:1 to about 11:5, and alternatively from about 6:5 to about 2:1.

The composition can comprise an emollient including at least one emollient or a combination of emollients. Suitable emollients are often liquid under ambient conditions. Depending on the type of product form desired, the amount of emollient(s) in the composition may be at least about 10%, by weight of the composition. In some embodiments, the composition may comprise one or more emollients from about 10% to about 80%, by weight of the composition, and in other embodiments, the composition may comprise one or more emollients, by weight of the composition, at least about 20%, at least about 25%, or from about 20% to about 80%, or from about 25% to about 80%, or even be 100%.

The composition may comprise an emulsion, either an oil-in-water emulsion or a water-in-oil emulsion. If there is an emulsion, the continuous phase may comprise one or more emollients.

The composition can be an oil-in-water emulsion that includes an oil phase that is dispersed into a continuous water phase. The oil phase may include an emollient, an emulsifier, and optionally other ingredients such as, but not limited to, co-emulsifiers, fragrances, deodorant actives, skin conditioners, thickeners, colloidal stabilizers, polymeric emulsifiers or other oil soluble ingredients. Emollients in the oil phase can be water insoluble liquids that smooth, soften, or lubricate the skin and will typically comprise more than 30% of an oil phase. The role of the oil phase in the composition can be multifold. An oil phase must be water insoluble enough to provide a stable emulsion, not interfere with the antiperspirant active, provide a solvent system for the fragrance, and provide a lubricious soft feel to the consumer's skin throughout the day.

The continuous phase of the oil-in-water emulsion may be comprised of water-soluble emollients, thickeners, colloidal stabilizers, polymeric emulsifiers, solubilizers, emulsifiers, deodorant actives, pre-biotics, pro-biotics, post-biotics, and antiperspirant actives. The actives and emollients need to be water soluble enough to form a stable emulsion.

In some cases, the product comprises a composition comprising an oil-in-water emulsion comprising a continuous phase, wherein the continuous phase comprises at least about 10%, by weight of the mixture, of one or more water-soluble emollients. In some cases, the continuous phase may comprise from about 10% to about 40%, by weight of the composition, of at least one water-soluble emollient, in other cases from about 15% to about 40%, from about 20% to about 40%, or from about 20% to about 40%, or from 10% to 50%.

The composition can be a water-in-oil emulsion that can include a water phase that is dispersed into a continuous oil phase and/or silicone phase. The water phase may comprise of one or more water soluble emollients, an emulsifier, and optionally other ingredients such as, but not limited to, co-emulsifiers, deodorant actives, skin conditioners, antiperspirant active and or other water-soluble ingredients. The actives and emollients need to be water soluble enough to form a stable emulsion.

The continuous phase of the water-in-oil emulsion is comprised of water insoluble emollients, liquids that smooth, soften, or lubricate the skin and will typically comprise more than 10-50% the composition. As per oil-in-water emulsion, the role of the oil phase in the water-in-oil composition is multifold. An oil phase must be water insoluble enough to provide a stable emulsion, not interfere with the antiperspirant active, provide a solvent system for the fragrance, and provide a lubricious soft feel to the consumer's skin throughout the day.

In some cases, such as when the composition is a single phase, all the emollients or at least one of the emollients may have a viscosity of at least about 20 cP. The higher viscosity emollients, such as those with viscosity of at least 20 cP, are thicker emollients and provide good consumer skin feel. For products with a single-phase composition, the one or more emollients may be water-soluble or water-insoluble or some combination thereof. In cases in which an emulsion comprises a water-insoluble emollient, at least one water-insoluble may have a viscosity of at least about 20 cP. In cases in which an emulsion comprises at least one water-soluble emollient, at least one emollient may have a viscosity of at least about 10 cP, in some cases at least about 5 cP.

### Emollients

Emollients can be either water soluble or water insoluble. Water soluble emollients are identified as able to form a single-phase mixture with water, with light transmission of the sample of greater than about 95%, as measured in the test method described herein. Water soluble emollients can include, but are not limited to, propylene glycol, polypropylene glycol (like dipropylene glycol, tripropylene glycol, etc.), diethylene glycol, triethylene glycol, PEG-4, PEG-8, PEG-6 Caprylic/Capric Glycerides, PEG-7 Glyceryl Cocoate, 1,2 pentanediol, 1,2 hexanediol, hexylene glycol, glycerin, C2 to C20 monohydric alcohols, C2 to C40 dihydric or polyhydric alcohols, water soluble alkyl ethers of polyhydric and monohydric alcohols. Water soluble emollients can also include polyglycerol based materials like polyglyceryl-10 heptanoate, and Polyglyceryl-2 Dipolyhydroxystearate. Water-insoluble emollients can include, but are not limited to, volatile silicone emollients such as cyclopentasiloxane, nonvolatile silicone emollients such as dimethicone, mineral oils, petrolatum, water insoluble alkyl ethers, esters, carbonates, low melting point triglycerides and combinations thereof. A suitable water insoluble emollient can comprise PPG-15 stearyl ether, isopropyl myristate, caprylic capric triglyceride, butyloctanol and dipropyl heptyl carbonate.

Solubility of an emollient in water may be determined by measuring the amount of light transmittance (a light transmittance value) through a simple mixture of water and emollient at the same weight/weight concentrations as in a final composition. For example, the solubility of an emollient at a concentration of 19% w/w in a final composition comprising water having a concentration of 38% w/w can be determined by measuring the light transmittance of an emollient at 19% w/w concentration in just water. Light transmittance may be measured using a spectrophotometer, such as, for example, a Genesys 10 Vis Spectrophotometer available from Thermo Electron Corp (USA), wherein a light transmittance value greater than 95% at 25° C. indicates sufficient solubility in water.

The composition may include one or more emollients that can be natural or naturally derived and/or biodegradable, alternatively all the emollients can be natural or naturally derived and/or biodegradable. The composition can be formulated without, free of, or substantially free of petroleum-based emollients. The emollient can be a liquid emollient having a melting point below 40° C., below 35° C., or below 30° C. The liquid emollient can be an oil, which can include an ester, alkane, triglyceride, non-volatile silicones, and combinations thereof. In another example, the emollient can be a waxy emollient, such as a fatty alcohol, having a melting point above 40° C., alternatively above 50° C. In some examples, the composition can contain one or more liquid emollients and one more waxy emollients.

The composition can include from about 1% to about 90%, alternatively from about 5% to about 70%, alternatively from about 5% to about 60% emollient, alternatively from about 5% to about 40%, and alternatively from about 10% to about 20% of one or more emollients.

The emollient can be a naturally derived oil that is a plant oil. Examples of the plant oil can include, but are not limited to, palm kernel, coconut, avocado, canola, corn, cottonseed, olive, palm, hi-oleic sunflower, mid-oleic sunflower, sunflower, palm stearin, palm kernel olein, safflower, babassu oils, and combinations thereof. In one embodiment, palm kernel oil may be the selected oil. In another embodiment, coconut oil may be the selected oil. In another embodiment, the plant oil may be a combination of palm kernel oil and coconut oil.

The composition can include one or more of the following liquid emollients:
An alkane or isoalkane, ranging in total carbon from about C8-C60, including but not limited to squalane, squalene, coconut alkanes, coconut/palm kernel alkanes, C9-12 Alkane, C10-13 Alkane, C13-15 Alkane, C15-19 Alkane, C14-22 Alkane, C30-34 Alkane, C9-20 Isoalkane, C10-12 Isoalkane, C12-15 Isoalkane, isohexadecene, C32-54 Isoalkane, or a combination thereof.
An ester formed from an alcohol having from 1-6 carbons and acid having from about 6-22 carbons, including but not limited to isopropyl isostearate, isopropyl palmitate, isoamyl laurate, shea butter ethyl esters, shea butter cetyl esters, shea butter decyl esters, shea butter oleyl esters, jojoba esters, coco-capylate/caprate, ethyl caprate, ethylhexyl caprate, isoamyl caprylate/caprate, argan oil isostearyl esters, babassu seed oil ethyl esters, camelina sative seed oil ethyl esters, castor/olive oil esters, coconut oil decyl esters, coconut oil ethyl esters, linseed oil ethyl esters, olive oil decyl esters, olive oil ethylhexyl esters, palm kernel oil triacetin esters, palmitoyl serine/silk amino acid methyl esters, rapeseed oil decyl esters, safflower seed oil decyl esters, sunflower seed oil cetyl esters, sunflower seed oil ethyl esters, trehalose isostearate esters, or a combination thereof.
A triglyceride having 3 carbon chains where the carbon chain length ranges from about C7-C18, including but not limited to triheptanoin, caprylic/capric triglyceride, C10-19 triglycerides, caprylic/capric/lauric triglycerides, hydrogenated C12-C18 triglycerides, triethylhexanoin, or a combination thereof.
A non-volatile silicone fluid including but not limited to dimethicone, amodimethicone, or a combination thereof.

### Emulsifiers, Surfactants and Co-Emulsifiers

The composition may also comprise one or more of the following: emulsifiers, and co-emulsifiers. These materials may perform several functions in the composition including, but not limited to, stabilizing an emulsion, solubilization of the actives and fragrance. Choice of these materials is dependent on the composition of the emulsion, particularly whether the composition is a water-in-oil or an oil-in-water emulsion. Moreover, it is desirable to choose materials that would not adversely impact surface tension by lowering it below the viscosity of the continuous phase. Furthermore, the choice of any emulsifier, surfactant, or co-emulsifier may not interfere with the performance of the antiperspirant or deodorant actives used in the antiperspirant or deodorant composition. For example, the use of some anionic surfactants can interfere with efficacy of cationic aluminum antiperspirant actives via the formation of insoluble ion pairs. Lastly, it is appreciated that different oil phase emollients will require different emulsifiers, surfactants, or co-emulsifiers to create a stable multiphase emulsion. Said differently, the different oil phases that are insoluble in one another may require different emulsifiers and co-emulsifiers to stabilize each emulsion phase in the emulsion.

An emulsifier or co-emulsifier used to stabilize an emulsion may be chosen based on the required HLB (hydrophilic lipophilic balance) of the oil phase emollients. The HLB of a surfactant is a measure of the ratio of the hydrophobic to the hydrophilic portion of the surfactant or emulsifier. Choice of the desired HLB for an emollient is dependent on the emollient or emollient blend polarity and structure. The use of the HLB system for emulsion formulation is discussed in the following references:
1. Griffin W C; Calculation of HLB Values of Non-Ionic Surfactants, Journal of the Society of Cosmetic Chemists; 1954. Vol. 5, pp 249-235
2. Vaughan, C. D. Rice, Dennis A.; Predicting O/W Emulsion Stability by the “Required HLB Equation”; Journal of Dispersion Science and Technology; 1990. Vol. 11 (1), pp 83-91.

Any known emulsifier, or co-emulsifier can be used in a composition intended for use as an aerosol antiperspirant or deodorant compositions herein, provided that they stabilize the emulsions and do not interfere with the delivery or action of the antiperspirant or deodorant actives. Suitable classes of emulsifiers and surfactants include anionic, cationic, and nonionic materials. Moreover, for some oil phase emollients, polymeric emulsifiers and surfactants are suitable. In some embodiments, nonionic emulsifiers, and co-emulsifiers are preferred to prevent interaction with any charged antiperspirant active (e.g., aluminum chlorohydrate) or deodorant active (e.g., benzethonium chloride).

Emulsifier, and co-emulsifier concentrations will vary based on composition, however it is generally found to be desirable to not have excess emulsifier and co-emulsifier to prevent lowering the surface tension of the continuous phase and increase particle size of spray. Total levels of emulsifier, surfactant, and co-emulsifier can be less ≤12%, ≤7% and ≤3%, by weight of the antiperspirant or deodorant composition for an oil-in-water emulsion. Total levels of emulsifier, surfactant, and co-emulsifier can be ≤5%, ≤3%, and ≤1%, by weight of the antiperspirant or deodorant composition for water-in-oil emulsion.

Suitable nonionic emulsifiers and co-emulsifier can include, but are not limited to, linear saturated and unsaturated C12 to C30 primary alcohols that are etherified with 1 to 100 ethylene oxide units per molecule. More preferred nonionic emulsifiers laureth, trideceth, myristeth, ceteth, ceteareth steareth, arachideth, and beheneth, having respectively 1 to 100 ethylene oxide units per molecule. Some examples of preferred nonionic emulsifiers include, but are not limited to, steareth-1, steareth-2, steareth-3, steareth-20, steareth-21, steareth-100, ceteareth-10. ceteareth-20 ceteareth-30, ceteth-1, ceteth-2, ceteth-3, ceteth-10, myristeth-1, myristeth-2, laureth-4, beheneth-2, beheneth-3, and beheneth-5, behenth-10, and beheneth-25.

The composition can include steareth-2 and steareth-21. The weight ratios of steareth-21 to steareth-2 can range from about 0.2 to about 5, alternatively from about 0.2 to about 2.

The composition can include ceteth-10 and laureth-4.

Suitable nonionic polymer emulsifiers and co-emulsifiers can include but are not limited to dimethicone copolymers, namely polyoxyalkylene modified dimethylpolysiloxanes. The polyoxyalkylene group can be a polyoxyethylene (POE) or polyoxypropylene (POP) or a copolymer of POE and POP. The copolymers can also include C1 to C12 alkyl groups as functional groups. Examples of suitable surfactants include DC5225 and DC 5200 (from Dow Corning), Abil EM 90 and EM 97 (from Gold Schmidt) and KF 6026, KF 6028, KF 6038 (from Shinetsu®).

Suitable cationic emulsifiers and surfactants can include, but are not limited to, distearyldimonium chloride, behentrimonium chloride and palmitamido-propyltrimonium chloride.

Suitable co-emulsifiers can include, but are not limited to, fatty alcohols such as stearyl alcohol, cetyl alcohol, and cetearyl alcohol.

In some examples, the emulsion may not comprise a fatty alcohol, as fatty alcohols can increase the viscosity, especially if the composition is an oil-in-water emulsion to a level that is difficult to spray, making them undesirable.

### Chelator

The compositions may comprise a chelant. Chelants may include, but are not limited to, diethylenetriaminepentaacetic acid (DTPA), diethylenetriaminepentakis (methylenephosphonic acid) (DTPMP), desferrioxamine, ethylenediaminetetraacetic acid (EDTA), ethylenediamine-N,N′-disuccinic acid (EDDS), enterobactin, desferrioxamine, HBED, trisodium dicarboxymethyl alaninate (MGDA), salts thereof and combinations thereof. The amount of chelant, by weight of composition, may be from about 0.05% to about 4%, alternatively from about 0.1% to about 1%, and alternatively from about 0.2% to about 0.5%.

### Preservatives

The composition can include a preservative. The preservative can be added to the product composition or with the liquid transfer agent. The preservative is included in an amount sufficient to prevent spoilage or prevent growth of inadvertently added microorganisms for a specific period of time. In other words, the preservative is not being used as the antimicrobial compound to kill microorganisms on the surface onto which the composition is deposited in order to eliminate odors produced by microorganisms. Instead, it is being used to prevent spoilage of the composition in order to increase shelf-life.

The preservative can be any organic preservative material which will not cause damage to fabric appearance, e.g., discoloration, coloration, bleaching. Suitable water-soluble preservatives include organic sulfur compounds, halogenated compounds, cyclic organic nitrogen compounds, low molecular weight aldehydes, parabens, propane diol materials, isothiazolinones, quaternary compounds, benzoates, low molecular weight alcohols, phenyl and phenoxy compounds, or mixtures thereof.

Non-limiting examples of commercially available water-soluble preservatives include a mixture of about 77% 5-chloro-2-methyl-4-isothiazolin-3-one and about 23% 2-methyl-4-isothiazolin-3-one, a broad spectrum preservative available as a 1.5% aqueous solution under the trade name Kathon® CG by Rohm and Haas Co.; 5-bromo-5-nitro-1,3-dioxane, available under the tradename Bronidox L® from Henkel; 2-bromo-2-nitropropane-1,3-diol, available under the trade name Bronopol® from Inolex; 1,1′-hexamethylene bis(5-(p-chlorophenyl)biguanide), commonly known as chlorhexidine, and its salts, e.g., with acetic and digluconic acids; a 95:5 mixture of 1,3-bis(hydroxymethyl)-5,5-dimethyl-2,4-imidazolidinedione and 3-butyl-2-iodopropynyl carbamate, available from Lonza; N-[1,3-bis(hydroxymethyl) 2,5-dioxo-4-imidazolidinyl]-N,N′-bis(hydroxy-methyl) urea, commonly known as diazolidinyl urea, available under the trade name Germall® II from Sutton Laboratories, Inc.; N,N″-methylenebis {N′-[1-(hydroxymethyl)-2,5-dioxo-4-imidazolidinyl]urea}, commonly known as imidazolidinyl urea, available, e.g., under the trade name Abiol® from 3V-Sigma, Unicide U-13® from Induchem, Germall 115® from Sutton Laboratories, Inc.; polymethoxy bicyclic oxazolidine, available under the trade name Nuosept® C from Hüls America; formal-dehyde; glutaraldehyde; polyaminopropyl biguanide, available under the trade name Cosmocil CQ® from ICI Americas, Inc., or under the trade name Mikrokill® from Brooks, Inc; dehydroacetic acid; and benzisothiazolinone available under the trade name Koralone™ B-119 from Rohm and Hass Corporation.

The preservative can be one or more water-soluble ingredients including, but not limited to sodium benzoate, potassium hydrogen caprylhydroxamate, iodopropynyl butyl carbamate, benzoic acid, phenozyethanol, citric acid, DMDM hydantoin, methylchloroisothiazolinone, methylisothiazolinone, or mixtures thereof. The preservative can be an ingredient with limited water solubility including, but not limited to methylparaben, propylparaben, hydroxyacetophenone, caprylhydroxamic acid, glyceryl heptanoate, 1,2-hexandiol, glyceryl caprylate, caprylyl glycol, or mixtures thereof. The preservative can be an ingredient that is not typically categorized as a preservative, but either alone or in combination with other ingredients prevents spoilage or prevent growth of inadvertently added microorganisms for a specific period.

Suitable levels of preservative can range from about 0.0001% to about 0.5%, alternatively from about 0.0002% to about 0.2%, alternatively from about 0.0003% to about 0.1%, by weight of the composition. Suitable levels of preservative can range from about 0.05% to about 1.5%, alternatively from about 0.1% to about 1.0%, alternatively from about 0.2% to about 0.5%, by weight of the composition.

### Solvents

As used herein, solvents are liquids intended to dissolve, break down, and/or disperse other ingredients in the formulation, examples of such include, but are not limited to, ethanol, water, isopropyl alcohol, polydecene, decane, isodecane, 1-decene, 1-heptanol, 1-hexanol, 1-hexene, 1-methoxy-2-propanaol acetate, 1-octene, 2,2,4-trimethylpentane, 2-butanone, 2-butoxyethanol, 2-ethoxyethnol.

For single phase products, the amount of solvent may range from about 0% to about 90%, by weight of the composition, or from about 20% to about 80%, from about 30%, about 40%, about 50% to about 55%, about 65%, or about 75%, by weight of the composition. For products with water-in-oil emulsions or oil-in-water emulsions, the continuous phase may comprise from about 0% to about 90% solvent, by weight of the composition, or from about 20% to about 80%, from about 30%, about 40%, about 50% to about 55%, about 65%, or about 75%, by weight of the composition.

### Fragrance

One or more fragrance materials are included to help cover or mask malodors resulting from perspiration, or which otherwise provide the compositions with the desired perfume aroma. These fragrance materials may include any perfume or perfume chemical suitable for topical application to the skin.

The concentration of the fragrance in the compositions may be effective to provide the desired aroma characteristics or to mask malodor wherein the malodor is inherently associated with the composition itself or is associated with malodor development from human perspiration. Compositions may comprise fragrances chosen from free perfumes, encapsulated perfumes, and mixtures thereof. The total perfume may include one or more individual perfume chemicals provided that the perfume can emit a detectable perfume odor or can mask or help to mask odors associated with perspiration. Generally, the compositions may comprise the total perfume at concentrations ranging from about 0.05% to about 10%, from about 0.5% to about 5% from about 1% to about 4%, or from about 1.5% to about 2.5%. As previously discussed, the choice of fragrance level and the emollient level both in the first oil phase are often related by desired emollient to fragrance weight ratios of from about 1:1 to about 10:1 or from 3:1 to about 7:1. The fragrance that is in the antiperspirant and/or deodorant composition may be entirely in the first oil phase and may be solubilized in the first oil phase.

Nonlimiting examples of fragrance materials suitable for use as a free perfume or an encapsulated perfume include any known fragrances in the art or any otherwise effective fragrance materials. Typical fragrances are described in Arctander, Perfume and Flavour Chemicals (Aroma Chemicals), Vol. I and II (1969) and Arctander, Perfume and Flavour Materials of Natural Origin (1960). U.S. Pat. No. 4,322,308, issued to Hooper et al., Mar. 30, 1982 and U.S. Pat. No. 4,304,679, issued to Hooper et al., Dec. 8, 1981 disclose suitable fragrance materials including, but not limited to, volatile phenolic substances (such as iso-amyl salicylate, benzyl salicylate, and thyme oil red), essence oils (such as geranium oil, patchouli oil, and petitgrain oil), citrus oils, extracts and resins (such as benzoin siam resinoid and opoponax resinoid), “synthetic” oils (such as Bergamot™ 37 and Bergamot™ 430, Geranium™ 76 and Pomeransol™ 314); aldehydes and ketones (such as B-methyl naphthyl ketone, p-t-butyl-A-methyl hydrocinnamic aldehyde and p-t-amyl cyclohexanone), polycyclic compounds (such as coumarin and beta-naphthyl methyl ether), esters (such as diethyl phthalate, phenylethyl phenylacetate, non-anolide 1:4).

Suitable fragrance materials may also include esters and essential oils derived from floral materials and fruits, citrus oils, absolutes, aldehydes, resinoides, musk and other animal notes (e.g., natural isolates of civet, castoreum and musk), balsamic, and alcohols (such as dimyrcetol, phenylethyl alcohol and tetrahydromuguol). For example, the composition may comprise fragrances selected from the group consisting of decyl aldehyde, undecyl aldehyde, undecylenic aldehyde, lauric aldehyde, amyl cinnamic aldehyde, ethyl methyl phenyl glycidate, methyl nonyl acetaldehyde, myristic aldehyde, nonalactone, nonyl aldehyde, octyl aldehyde, undecalactone, hexyl cinnamic aldehyde, benzaldehyde, vanillin, heliotropine, camphor, para-hydroxy phenolbutanone, 6-acetyl 1,1,3,4,4,6 hexamethyl tetrahydronaphthalene, alpha-methyl ionone, gamma-methyl ionone, amyl-cyclohexanone, and mixtures thereof.

### Actives

The product composition may be an antiperspirant or deodorant composition. Antiperspirant products reduce sweating or perspiration on the skin, typically by temporarily blocking the pores and reducing the amount of sweat that reaches the surface. Deodorants work by combating the bacteria on the skin that break down sweat, which is what causes body odor. Antiperspirants can include deodorant actives and both antiperspirants and deodorants can include fragrances to mask and improve body odor.

If the product composition is an emulsion, the active is generally dissolved in the water phase. The concentration of the active in the composition should be sufficient to provide the finished antiperspirant or deodorant composition with the desired perspiration wetness and/or odor control benefits.

Exemplary antiperspirant active concentrations range include from about 0.1% to about 26%, from about 1% to about 20%, and from about 2% to about 10%, by weight of the composition. All such weight percentages are calculated on an anhydrous metal salt basis exclusive of water and any complexing or buffering agent such as, for example, glycine, glycine salts or other amino acids and any stabilizing agents such as calcium chloride, calcium salts, sodium salts or strontium salts.

Aluminum salts can have the general formula Al₂(OH)₆₋ₐXₐ wherein X is Cl, Br, I, or NO₃, and a is about 0.3 to about 5, alternatively from about 0.8 to about 2.5. Preferred actives in this group include, but are not limited to, aluminum chlorohydrate (ACH) wherein a is from about 1 and the mole ratio of Al/Cl is from about 1.9 to about 2.1, Aluminum sesquichlorohydrate (ASCH) wherein a is from about 1.05 to about 1.61 and the mole ratio of Al/Cl is from about 1.26 to about 1.89, and aluminum dichlorohydrate (ADCH) wherein a is from about 1.6 to about 2.2 and the mole ratio of Al/Cl is from about 0.9 to about 1.25.

The ACH salts may also include soluble calcium salts. Soluble calcium salts are those calcium salts that are soluble in water or that dissolve in the aqueous solution of antiperspirant salt (i.e., a solution of the aluminum salt and/or zirconium salt). Calcium salts which may be utilized are any of those which do not otherwise interfere with the solubility or effectiveness of the antiperspirant salt. Preferred calcium salts include calcium chloride, calcium bromide, calcium nitrate, calcium citrate, calcium formate, calcium acetate, calcium gluconate, calcium ascorbate, calcium lactate, calcium glycinate and mixtures thereof. Calcium carbonate, calcium sulfate and calcium hydroxide may also be used because they will dissolve in an aqueous solution of the antiperspirant salt.

The ACH salts may also include soluble sodium salts. Soluble sodium salts are those sodium salts that are soluble in water or that dissolve in the aqueous solution of antiperspirant salt (i.e., a solution of the aluminum salt and/or zirconium salt). Sodium salts which may be utilized are any of those which do not otherwise interfere with the solubility or effectiveness of the antiperspirant salt. Sodium salts can include sodium chloride, sodium nitrate, and mixtures thereof. Sodium sulfate may also be used because they will dissolve in an aqueous solution of the antiperspirant salt.

The ACH salt can be chosen from sodium chloride, potassium chloride, lithium chloride, or mixtures thereof.

Additional ACH actives are disclosed in U.S. Prov. Pat. App. 63/702,962, incorporated by reference.

The ACH salts used may also contain a water soluble amino and/or hydroxy acid which is effective in increasing and/or stabilizing the HPLC peak 4:3 area ratio of the antiperspirant salt. Such acids include amino- and/or hydroxy-substituted lower alkanoic acids (including substituted derivatives thereof), preferably where the amino or hydroxy group is located on the α-carbon (i.e., the same carbon to which the carboxy group is attached). The lower alkanoic acid will generally have 2 to 6, preferably 2 to 4, carbon atoms in the alkanoic acid chain. Typical amino and/or hydroxy substituted lower alkanoic acids include any of the amino acids such as glycine, alanine, valine, leucine, isoleucine, P-alanine, serine, cysteine, β-amino-n-butyric acid, γ-amino-n-butyric acid, etc. and hydroxy acids such as glycolic acid and lactic acid. These amino and/or hydroxy substituted lower alkanoic acids may also contain various substituents which do not adversely affect their activity. The preferred amino and/or hydroxy substituted lower alkanoic acids are glycine, alanine, and glycolic acid, with glycine being most preferred. The active can include an aqueous solution of ACH that contains calcium chloride, sodium chloride and glycine. The ACH can have a molar ratio of glycine to calcium of less than 2, alternatively less than or equal to 1.75, alternatively less than or equal to 1.5. The ACH can have a pH of about 4.2 to about 5.3, alternatively from about 4.5 to about 5.0. The ACH can have a peak IV/III ratio exceeding 0.75 as measured by HPLC.

The active can be an aqueous solution of ADCH that also contains calcium chloride and glycine. The preferred ADCH with calcium chloride and glycine is further characterized by having more than 50% peak 4 and 5 as measured by HPLC, a Al:Cl molar ratio of about 0.9 to about 1.25, an Al to glycine wt. ratio of about 1.7 to 7.7, and calcium to glycine wt. ratio of about 0.1 to about 1.5. The active can be an aqueous solution of ASCH that also contains calcium chloride and glycine. The preferred ASCH with calcium chloride and glycine is further characterized by having more than 35% peak 4 and 5 as measured by HPLC, a Al:Cl molar ratio of about 1.26 to about 1.89, an Al to glycine wt ratio of about 4 to 10, and calcium to glycine ratio of about 0.1 to about 1.5.

The antiperspirant or deodorant compositions provided herein may comprise a non-aluminum antiperspirant active. Suitable non-aluminum antiperspirant actives include, but are not limited to, oxybutynin chloride, chitosan, PVM/MA polymers, calcium channel blockers, gingerol, liquid fatty acid and metal ion combinations, magnesium gluconate, silicic acid, silicic acid salts, and vicinal diols such as propylene glycol.

The compositions may comprise a deodorant active. The deodorant active can be used instead of an antiperspirant active or in addition an antiperspirant active. Some deodorants may not have an antiperspirant active and/or may be substantially free or free of aluminum. Suitable deodorant actives may be chosen from antimicrobial agents (e.g., bactericides, fungicides), malodor-absorbing material, and combinations thereof. For example, antimicrobial agents may comprise cetyl-trimethylammonium bromide, cetyl pyridinium chloride, benzethonium chloride, diisobutyl phenoxy ethoxy ethyl dimethyl benzyl ammonium chloride, sodium N-lauryl sarcosine, sodium N-palmethyl sarcosine, lauroyl sarcosine, N-myristoyl glycine, potassium N-lauryl sarcosine, trimethyl ammonium chloride, sodium aluminum chlorohydroxy lactate, triethyl citrate, tricetylmethyl ammonium chloride, 2,4,4′-trichloro-2′-hydroxy diphenyl ether (triclosan), 3,4,4′-trichlorocarbanilide (triclocarban), diaminoalkyl amides such as L-lysine hexadecyl amide, heavy metal salts of citrate, salicylate, and piroctose, especially zinc salts, and acids thereof, heavy metal salts of pyrithione, especially zinc pyrithione, zinc phenolsulfate, farnesol, and combinations thereof.

The deodorant active may include an antibacterial chosen from 2-Pyridinol-N-oxide (piroctone olamine), lupamin, beryllium carbonate, magnesium carbonate, calcium carbonate, magnesium hydroxide, magnesium hydroxide and magnesium carbonate hydroxide, partially carbonated magnesium hydroxide, potassium carbonate, potassium bicarbonate, sodium carbonate, sodium sesquicarbonate, baking soda, hexamidine, zinc carbonate, thymol, polyvinyl formate, salycilic acid, niacinamide and combinations thereof.

The concentration of the deodorant active may range, individually or cumulatively, from about 0.001%, from about 0.01%, of from about 0.1%, by weight of the composition to about 20%, to about 10%, to about 5%, or to about 1%, by weight of the composition.

### Odor Entrappers

The composition can include an odor entrapper. Suitable odor entrappers can include solubilized, water-soluble, uncomplexed cyclodextrin. As used herein, the term “cyclodextrin” includes any of the known cyclodextrins such as unsubstituted cyclodextrins containing from six to twelve glucose units, especially, alpha-cyclodextrin, beta-cyclodextrin, gamma-cyclodextrin and/or their derivatives and/or mixtures thereof. The alpha-cyclodextrin consists of six glucose units, the beta-cyclodextrin consists of seven glucose units, and the gamma-cyclodextrin consists of eight glucose units arranged in a donut-shaped ring. The specific coupling and conformation of the glucose units give the cyclodextrins a rigid, conical molecular structure with a hollow interior of a specific volume. The “lining” of the internal cavity is formed by hydrogen atoms and glycosidic bridging oxygen atoms; therefore, this surface is fairly hydrophobic. The unique shape and physical-chemical property of the cavity enable the cyclodextrin molecules to absorb (form inclusion complexes with) organic molecules or parts of organic molecules which can fit into the cavity. Many perfume molecules can fit into the cavity.

Cyclodextrin molecules are described in U.S. Pat. Nos. 5,714,137, and 5,942,217. Suitable levels of cyclodextrin are from about 0.1% to about 5%, alternatively from about 0.2% to about 4%, alternatively from about 0.3% to about 3%, alternatively from about 0.4% to about 2%, by weight of the composition.

### Methods of Use

A user of an aerosol product may initiate a spray by depressing an actuator. While usage time can vary widely, users of an aerosol product may depress the actuator from about 2 seconds to about 5 seconds, or from about 2 seconds to about 4 seconds, or from about 2 seconds to about 3 seconds to provide a burst of deodorant concentrate for deposition to an underarm skin surface. The product can be sprayed from 2 inches (5.08 cm) to 12 inches (30.48 cm), 3 inches (7.62 cm) to 10 inches (25.4 cm), 4 inches (10.16 cm) to 8 inches (20.32 cm), or 6 inches (15.24 cm) away from target area.

The aerosol product can be a deodorant and/or antiperspirant composition, which can include a whole body deodorant. The product can be used on various areas of the body, including underarms, chest, legs, stomach, groin, privates, underboobs, neck, feet, arms, hands, and more. The deodorant and/or antiperspirant can be a leave-on composition that can go on clear and can form an invisible layer of protection, ensuring no residue. The deodorant and/or antiperspirant can make user's smell great and keep them feeling fresh and dry all day long. The deodorant and/or antiperspirant product can provide 72 hours of freshness and even lasts when it is 100° F. The product can help maintain a healthy, balanced, pH for a user's skin. The product can be gentle on skin. The product can fight odor causing bacteria. The product can be a low ethanol product, which can be applied to sensitive skin.

### TEST METHODS

### Spray Rate

Spray rate was determined by measuring the weight loss of the canister after 5 seconds of complete actuation of the dispenser, this process is repeated with approximately 5 minutes between sprays, with the canister being shaken in a vertical motion 6 times prior to each spray. Spray rates are determined until the canister is no longer able to eject an appreciable amount of liquid, generally defined as spray rates less than 0.05 g/sec. The reported spray rate at each measurement is the mass lost divided by the spray time of that actuation event. Each spray rate is reported at the total spray time the data point was collected at, which is the summation of time the actuator has been depressed at the end of that spray event and prior spray events.

### EXAMPLES

The following examples are given solely for the purpose of illustration and are not to be construed as limitations of the invention as many variations thereof are possible without departing from the spirit and the scope of the invention.

TABLE 16
Actuator Examples

| | Actuator A | Actuator B | Actuator C |
|---|---|---|---|
| Exit Orifice Diameter (mm) | 0.3 | 0.25 | 0.25 |
| Length of Exit Orifice (mm) | 0.2 | 0.25 | 0.15 |

TABLE 17
Connector Examples

| | Connector A | Connector B |
|---|---|---|
| Liquid Orifice Diameter (mm) | 0.2 | 0.2 |
| Gas Orifice Diameter (mm) | 0.2 | 0.2 |
| Number of Gas Orifices | 1 | 2 |

In, Composition Examples 1-3 are homogenous single-phase solutions with Newtonian viscosities and Composition Example 4 is an oil-in-water emulsion with a non-Newtonian viscosity. The composition examples can be made by any known method that creates single-phase or oil-in-water emulsions. Composition Examples 1-4 are intended for use as deodorant compositions that are typically applied topically to the underarms or other areas of the body prone to sweating including chest including underboob, legs including thighs, stomach, feet, arms, hands, and privates.

TABLE 18
Product Composition Examples 1-4

| | Comp. | Comp. | Comp. | Comp. |
|---|---|---|---|---|
| | Ex. 1 | Ex. 2 | Ex. 3 | Ex. 4 |
| | (wt. %) | (wt. %) | (wt. %) | (wt. %) |
| Ethanol | Q.S. | | | 30 |
| Caprylic/Capric | 12.00 | | | 20 |
| Triglyceride | | | | |
| Dipropylene Glycol | 14.00 | 67.52 | 61.0 | |
| Propylene glycol | | 24.50 | | |
| Water | 5 | Q.S. | Q.S. | Q.S. |
| Citric Acid | | | 1.0 | 0.35 |
| Triethylcitrate | | | 1.0 | 1 |
| Piroctone olamine | | 0.10 | 0.1 | 0.10 |
| Polysorbate 20 | | | | 0.30% |
| Sodium Magnesium | | | | 0.50% |
| Fluorosilicate | | | | |
| Xanthan Gum | | | | 0.05% |
| Fragrance | 1.50 | 2 | 2 | 2 |

### Definitions

The term “substantially free of” refers to an amount of a material that is less than 1%, 0.5%, 0.25%, 0.1%, 0.05%, 0.01%, or 0.001% by weight of an antiperspirant composition. “Free of” refers to no detectable amount of the stated ingredient or thing.

The term “viscosity” means dynamic viscosity (measured in centipoise, cPs, or Pascal-second, Pa·s) or kinematic viscosity (measured in centistokes, cst, or m²/s) of a liquid at approximately 25° C. and ambient conditions. Dynamic viscosity may be measured using a rotational viscometer, such as a Brookfield Dial Reading Viscometer Model 1-2 RVT available from Brookfield Engineering Laboratories (USA) or other substitutable model known in the art. Typical Brookfield spindles which may be used include, without limitation, RV-7 at a spindle speed of 20 rpm, recognizing that the exact spindle may be selected as needed by one skilled in the art. Kinematic viscosity may be determined by dividing dynamic viscosity by the density of the liquid (at 25° C. and ambient conditions), as known in the art.

All percentages are by weight of the cosmetic composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 21° C. and at ambient conditions, where “ambient conditions” means conditions under about 1 atmosphere of pressure and at about 50% relative humidity. All weights as they pertain to listed ingredients are based on the active level and do not include carriers or by-products that may be included in commercially available materials, unless otherwise specified. All numeric ranges are inclusive of narrower ranges; delineated upper and lower range limits are interchangeable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

### Combinations

A. An aerosol product comprising an aerosol dispenser comprising:
   a. a dispenser container comprising a cavity; wherein an adsorbent, a transfer agent, and a compressed gas propellant are disposed within the cavity;
   b. a product reservoir disposed within the cavity;
   c. a product composition disposed within the product reservoir;
   d. a valve assembly; wherein the valve assembly comprises:
      i. a connector disposed in a lower end of the valve assembly; wherein the actuator is in fluid communication with the product reservoir and the cavity;
      ii. an actuator disposed in an upper end of the valve assembly; wherein the actuator is in operative communication with the connector;
   e. a filter for mitigating escape of the adsorbent from the container.
B. The aerosol product according to paragraph A, wherein the dispenser comprises a spray rate greater than 0.2 g/s for ≥50 s, preferably greater than 0.2 g/s for ≥70 s, more preferably greater than 0.2 g/s for ≥100 s, and even more preferably greater than 0.2 g/s for ≥110 s, according to the Spray Rate Method.
C. The aerosol product according to paragraphs A-B, wherein the dispenser comprises a spray rate greater than 0.1 g/s for ≥100 s, preferably greater than 0.1 for g/s ≥120 s, more preferably greater than 0.1 g/s for ≥130 s, and even more preferably greater than 0.1 g/s for ≥150 s, according to the Spray Rate Method.
D. The aerosol product according to paragraphs A-C, wherein the dispenser comprises a spray rate greater than 0.05 g/s for ≥125 s, preferably greater than 0.05 g/s for ≥140 s, more preferably greater than 0.05 g/s for ≥150 s, even more preferably greater than 0.05 g/s for ≥160 s, according to the Spray Rate Method.
E. The aerosol product according to paragraphs A-D, wherein the dispenser comprises a fill pressure of from about 45 psi (310 kPa) to about 180 psi (1241 kPa), preferably from about 60 psi (414 kPa) to about 125 psi (862 kPa), and more preferably from about 75 psi (517 kPa) to about 100 psi (689 kPa).
F. The aerosol product according to paragraph A-E, wherein the connector comprises a liquid orifice having an area and a gas orifice having an area and the ratio of the area of the liquid orifice to the area of the gas orifice is ≤1:1, preferably ≤3:4, more preferably ≤2:3, and even more preferably ≤1:2.
G. The aerosol product according to paragraph A-F, wherein the connector comprises a liquid orifice having an area and a gas orifice having an area and the ratio of the area of the liquid orifice to the area of the gas orifice is ≥1:5, preferably ≥1:4, and more preferably ≥1:3.
H. The aerosol product according to paragraph A-G, wherein the cavity has an inner wall and there is a lining disposed on at least a portion of the inner wall, and preferably substantially all of the inner wall.
I. The aerosol product according to paragraph H, wherein the liner is made from a material chosen from epoxy resin, polyethylene-based materials, polyamide-based materials, plant-based materials, or mixtures thereof.
J. The aerosol product according to paragraphs A-I, wherein the dispenser canister comprises about 2.5 g to about 120 g of the adsorbent, preferably from about 15 g to about 100 g of the adsorbent, more preferably from about 30 g to about 80 g of the adsorbent, and even more preferably from about 30 g to about 60 g of the adsorbent.
K. The aerosol product according to paragraphs A-I, wherein the dispenser canister comprises about 5 g to about 150 g of the adsorbent, preferably from about 20 g to about 140 g of the adsorbent, more preferably from about 40 g to about 120 g of the adsorbent, and even more preferably from about 60 g to about 100 g of the adsorbent.
L. The aerosol product according to paragraphs A-K, wherein the compressed gas is chosen from compressed air, oxygen, nitrogen, carbon dioxide, or mixtures thereof.
M. The aerosol product according to paragraph L, wherein the compressed gas is carbon dioxide.
N. The aerosol product according to paragraphs A-M, wherein the dispenser canister comprises ≥10 g of the adsorbent, preferably ≥20 g of the adsorbent, more preferably ≥30 g of the adsorbent, even more ≥60 g of the adsorbent, and most preferably ≥90 g of the adsorbent.
O. The aerosol product according to paragraphs A-N, wherein the adsorbent is chosen from activated carbon, zeolites, metal-organic frameworks, porous silica, amine-functionalized materials, biochar, graphene-based materials, or mixtures thereof.
P. The aerosol product according to paragraph O, wherein the adsorbent is activated carbon.
Q. The aerosol product according to paragraphs A-P, wherein the product reservoir comprises about 20 g to about 250 g of the product composition, preferably from about 30 g to about 200 g of the product composition, and more preferably from about 35 g to about 100 g of the product composition.
R. The aerosol product according to paragraphs A-Q, wherein transfer agents is a liquid at 20 to 25° C. and pressure from atmospheric pressure up to 180 psi (1241 kPa).
S. The aerosol product according to paragraphs A-R, wherein the transfer agents has a gas solubility of ≥0.5 g/L at 25° C., preferably ≥0.75 g/L at 25° C., more preferably ≥1 g/L at 25° C., and even more preferably ≥1.3 g/L at 25° C.
T. The aerosol product according to paragraphs A-S, wherein the transfer agent is chosen from glycerin, polyethylene oxide, propanediol, butylene glycol, dipropylene glycol, propylene glycol, water, silicone oil, liquid emollient, or mixtures thereof.
U. The aerosol product according to paragraphs A-T, wherein the transfer agent is water.
V. The aerosol product according to paragraphs A-U, wherein about 2 g to about 60 g of the transfer agent is disposed in the cavity, preferably from about 12 g to about 25 g of the transfer agent is disposed in the cavity, and more preferably from about 15 g to about 25 g of the transfer agent is disposed in the cavity.
W. The aerosol product according to paragraphs A-V, wherein ≥5 g of the transfer agent is disposed in the cavity, preferably alternatively ≥10 g of the transfer agent is disposed in the cavity, and more preferably ≥15 g of the transfer agent is disposed in the cavity.
X. The aerosol product according to paragraphs A-W, wherein the weight ratio of the transfer agent to adsorbent is ≥1:5, preferably ≥1:4, more preferably ≥1:3, and even more preferably ≥1:2.
Y. The aerosol product according to paragraphs A-X, wherein the weight ratio of the transfer agent to adsorbent is ≤1:1, preferably ≤7:8, more preferably ≤5:6, even more preferably ≤4:5, and most preferably ≤3:4.
Z. The aerosol product according to paragraphs A-Y, wherein the weight ratio of the transfer agent to adsorbent is from about 1:6 to less than 1:1, preferably from about 1:5 to about 3:4, more preferably from about 1:4 to about 2:3, even more preferably from about 1:4 to about 1:2, and most preferably from about 1:3 to about 1:2.
AA. The aerosol product according to paragraphs A-Z, wherein a preservative, antioxidant, and/or chelant is disposed within the cavity.
BB. The aerosol product according to paragraphs A-AA, wherein product compositions is chosen from a deodorant and/or antiperspirant, hairspray, dry shampoo, sunscreen, fabric care compositions, air care compositions, hard and soft surface care compositions, dusting spray, furniture polish, pesticides, herbicides, cooking spray, or mixtures thereof.
CC. The aerosol product according to paragraphs A-BB, wherein the dispenser container comprises a volume of from about 100 cm³ to about 500 cm³, preferably from about 150 cm³ to about 300 cm³, and even more preferably from about 170 cm³ to about 230 cm³.
DD. The aerosol product according to paragraphs A-CC, wherein a dispensing event dispenses a spray that comprises a plurality of particles comprising a Dv50 of from about 20 microns to about 140 microns, preferably from about 30 microns to about 60 microns.
EE. The aerosol product according to paragraphs A-DD, wherein a dispensing event dispenses a spray that comprises a plurality of particles comprising a Dv90 of from about 60 microns to about 140 microns, preferably from about 60 microns to 90 microns.
FF. The aerosol product according to paragraphs A-EE, wherein a dispensing event dispenses a spray that comprises a plurality of particles wherein less than 15% of the plurality of particles are under 10 microns, preferably less than 10% of the plurality of particles are under 10 microns, and preferably less than 5% of the plurality of particles are under 10 microns.
GG. The aerosol product according to paragraphs A-FF, wherein the product composition is a deodorant and/or antiperspirant composition.
HH. The aerosol product according to paragraph GG, wherein the deodorant and/or antiperspirant composition is a single phase composition.
II. The aerosol product according to paragraph GG-HH, wherein the deodorant and/or antiperspirant composition is a dual phase composition, preferably an emulsion.
JJ. The aerosol product according to paragraphs GG-II, wherein the product composition and the deodorant composition is substantially free of an aluminum containing active, preferably free of an aluminum containing active.
KK. The aerosol product according to paragraphs GG-JJ, wherein the product composition comprises an antiperspirant active.
LL. The aerosol product according to paragraph KK, wherein the product composition comprises from about 0.1% to about 26% of the antiperspirant active, preferably from about 1% to about 20% of the antiperspirant active, and more preferably from about 2% to about 10% of the antiperspirant active.
MM. The aerosol product according to paragraphs KK-LL, wherein the antiperspirant active is chosen from aluminum chlorohydrate, aluminum sesquichlorohydrate, aluminum dichlorohydrate or mixtures thereof.
NN. The aerosol product according to paragraphs GG-MM, wherein the product composition comprises from about 1% to about 90% of an emollient, preferably from about 5% to about 70% of an emollient, even more preferably from about 5% to about 60% of an emollient, and even more preferably from about 10% to about 40% of an emollient.
OO. The aerosol product according to paragraphs NN, wherein the emollient is chosen from a water soluble emollient, a liquid emollient, a plant oil, or mixtures thereof.
PP. The aerosol product according to paragraphs A-OO, having a weight ratio of adsorbent to product composition of at least 5:9, preferably at least 4:5, more preferably at least 1:1, more preferably at least 6:5, more preferably at least 7:5, more preferably at least 3:2, more preferably at least 7:4, and even more preferably at least 2:1.
QQ. The aerosol product according to paragraphs A-PP, having a weight ratio of adsorbent to product composition from about 3:5 to about 3:1, preferably from about 4:5 to about 5:2, more preferably from about 1:1 to about 11:5, and even more preferably from about 6:5 to about 2:1.
RR. A method of topical application of a composition comprising the use the aerosol product according to Paragraphs AA-QQ.
SS. The method according to paragraph RR, wherein the weight ratio of the compressed gas propellant to the product composition at an exit orifice is from about 1:40 to about 1:1, preferably from about 1:30 to about 1:2, and even more preferably from about 1:20 to about 1:3.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An aerosol product comprising an aerosol dispenser comprising:
a. a dispenser container comprising a cavity; wherein an adsorbent, a transfer agent, and a compressed gas propellant are disposed within the cavity;
b. a product reservoir disposed within the cavity;
c. a product composition disposed within the product reservoir;
d. a valve assembly; wherein the valve assembly comprises:
i. a connector disposed in a lower end of the valve assembly; wherein the actuator is in fluid communication with the product reservoir and the cavity;
ii. an actuator disposed in an upper end of the valve assembly; wherein the actuator is in operative communication with the connector;
e. a filter for mitigating escape of the adsorbent from the container.

2. The aerosol product of claim 1, wherein the dispenser comprises:
a. a spray rate greater than 0.2 g/s for ≥50 s;
b. a spray rate greater than 0.1 g/s for ≥100 s; and
c. a spray rate greater than 0.05 g/s for ≥125 s.

3. The aerosol product of claim 1, wherein the connector comprises a liquid orifice having an area and a gas orifice having an area and the ratio of the area of the liquid orifice to the area of the gas orifice is less than or equal to 1:1 and greater than or equal to 1:5.

4. The aerosol product of claim 1, wherein the adsorbent is chosen from activated carbon, zeolites, metal-organic frameworks, porous silica, amine-functionalized materials, biochar, graphene-based materials, or mixtures thereof.

5. The aerosol product of claim 4, wherein the dispenser canister comprises about from about 30 g to about 120 g of the adsorbent.

6. The aerosol of claim 5, wherein the adsorbent is activated charcoal.

7. The aerosol product of claim 1, wherein the compressed gas is chosen from compressed air, oxygen, nitrogen, carbon dioxide, or mixtures thereof.

8. The aerosol product of claim 7, wherein the compressed gas is carbon dioxide.

9. The aerosol product of claim 1, wherein the product reservoir contains 30 g to about 200 g of the product composition.

10. The aerosol product of claim 1, wherein transfer agents is a liquid at 20 to 25° C. and pressure from atmospheric pressure up to 180 psi.

11. The aerosol product of claim 10, wherein the transfer agents has a gas solubility of ≥0.5 g/L at 25° C.

12. The aerosol product of claim 1, wherein the transfer agent is chosen from glycerin, polyethylene oxide, propanediol, butylene glycol, dipropylene glycol, propylene glycol, water, silicone oil, liquid emollient, or mixtures thereof.

13. The aerosol product of claim 12, wherein the transfer agent is water.

14. The aerosol product of claim 1, wherein about 12 g to about 25 g of the transfer agent is disposed in the cavity.

15. The aerosol product of claim 1, wherein the weight ratio of the transfer agent to adsorbent is greater than or equal to 1:5 and less than or equal to 1:1.

16. The aerosol product of claim 1, wherein product compositions is chosen from a deodorant and/or antiperspirant, hairspray, dry shampoo, sunscreen, fabric care compositions, air care compositions, hard and soft surface care compositions, dusting spray, furniture polish, pesticides, herbicides, cooking spray, or mixtures thereof.

17. The aerosol product of claim 16, wherein the product composition is a deodorant and/or antiperspirant.

18. The aerosol product of claim 1, wherein a dispensing event dispenses a spray that comprises a plurality of particles comprising a Dv50 of from about 20 microns to about 140 microns.

19. A method of topical application of the product composition comprising the use the aerosol product according to claim 1.

20. The method of claim 19, wherein the weight ratio of the compressed gas propellant to the product composition at an exit orifice is from 1:20 to about 1:3.
